(19) United States
(12) Patent Application Publication
(10) Pub. No.: US 2025/0041221 A1
(43) Pub. Date: Feb. 6, 2025

(54) NANOPARTICLES FOR RELEASE OF NUCLEIC ACIDS

(71) Applicants: Universite Claude Bernard Lyon 1, Villeurbanne (FR); Centre National de la Recherche Scientifique, Paris (FR); Hospices Civils De Lyon, Lyon (FR); INSERM (Institut National de la Santé et de la Recherche Médicale), Paris (FR); Universita Degli Studi Di Verona, Verona (IT)

(72) Inventors: Giovanna Lollo, Lyon (FR); Valentina Andretto, Lyon (FR); David Kryza, Saint Didier au Mont d'or (FR); Eyad Al Mouazen, Lyon (FR); Mathieu Repellin, Lyon (FR); Stéphanie Briancon, Meyzieu (FR); Laurent Schaeffer, Francheville (FR); Jacquier Arnaud, Rillieux-la-Pape (FR); Laurent Coudert, Caluire (FR)

(73) Assignees: Universite Claude Bernard Lyon 1, Villeurbanne (FR); Centre National de la Recherche Scientifique, Paris (FR); Hospices Civils De Lyon, Lyon (FR); INSERM (Institut National de la Santé et de la Recherche Médicale), Paris (FR); Universita Degli Studi Di Verona, Verona (IT)

(21) Appl. No.: 18/715,327

(22) PCT Filed: Dec. 2, 2022
(86) PCT No.: PCT/EP2022/084174

§ 371 Date: Jun. 3, 2024

(30) Foreign Application Priority Data
Dec. 3, 2021 (FR) FR2112931

(51) Int. Cl. A61K 9/127; A61K 9/51; A61K 31/7105; A61K 31/711

(52) U.S. Cl. CPC A61K 9/1272; A61K 9/5123; A61K 9/5161; A61K 31/7105; A61K 31/711

(57) ABSTRACT

The invention relates to a nanoparticle comprising at least one cationic lipid, at least one neutral lipid, and at least one pH sensitive lipid which differs from said cationic lipid, which can be used as a vector for the transport and release of nucleic acids within cells.

The present invention relates to novel vectors for the intracellular administration of nucleic acids in in vitro or ex vivo models, or for therapeutic applications in vivo.

### PRIOR ART

Single- and double-stranded nucleic acids (antisense oligonucleotides, small interfering RNA, plasmid DNA and, more recently, mRNA) typically require a release system to reach their targets intact and efficiently, which system may be physical (electroporation, ultrasound-mediated delivery) or by means of nanometer-scale vectors. The vectors which are commonly used and are most effective at causing the nucleic acids to penetrate into a cell's cytoplasm are cationic chemical molecules of lipid or polymer type which, because of their charge, associate with the nucleic acid and allow it to enter the cell. These molecules, which are permanently charged or become positively charged in response to the pH of their environment, are very commonly used to cause nucleic acids (small or large DNA or RNA) to penetrate into cells in culture or cells in an organism. While they are highly effective in cultured cells, they are less so in organisms due to phenomena of being trapped by proteins and cells present in the blood. Prior-art patent EP 2389158 describes a composition based on an anionic molecule, which may for example be an alginic acid, and on a cationic lipid. This composition, which also comprises cholesterol, enables the release of a nucleic acid which is smaller than 200 nucleotides in size. It is also known to deliver nucleic acids by liposomes formed of combinations of DOTAP and DOPE, and cholesterol, such as in patent application WO2013/143555 from BioNTech, which liposomes are optionally covered with hyaluronic acid (HA). See Gasperini, Langmuir 31.11 (2015): 3308-3317; Hattori, et al, Journal of drug targeting 21.7 (2013): 639-647; Ruponen et al, Glycobiology and extracellular matrices, 276 (2001): 33875-33880.

### SUMMARY OF THE INVENTION

The inventors are now proposing novel nanoparticles which are both stable and enable targeted intracellular release of the nucleic acid.

The nanoparticles of the invention comprise
(a) at least one cationic lipid,
(b) at least one neutral lipid, and
(c) at least one pH-sensitive lipid which differs from the cationic lipid (a).

It will be appreciated that each type of lipid, (a), (b) and (c), can consist of a single lipid or of a mixture of a plurality of lipids of the same type, (a), (b), and (c), respectively.

In a particular embodiment, the nanoparticle is loaded with a nucleic acid.

Such a nanoparticle is of use for the intracellular delivery of said nucleic acid.

Preferably, the nanoparticle of the invention is covered by at least one anionic polysaccharide, preferably hyaluronic acid.

Advantageously, the nanoparticle of the invention is typically devoid of cholesterol, or even of any steroid.

### FIGURES

FIG. 1 is a schematic depiction of uncoated lipoplex complexes (LRC) and lipoplex complexes coated with hyaluronic acid (HLRC).

FIG. 2 shows cryoTEM images of liposomes. FIG. 2A shows the conventional morphology of unilamellar liposomes without mRNA. The interactions of the liposomes with mRNA and with mRNA/HA are shown in FIGS. 2B and 2C, respectively.

FIG. 3 reports the results in terms of transfection efficiency, cell viability and internalization of THP-1 cells after transfection with liposomes (DOTAP/DOPE/Coatsome SS-M; production by hydrating the lipid film), complexed with eGFP-mRNA at different N/P ratios. Flow cytometry analysis of transfection efficiency and fluorescence intensity of THP-1 monocytes with A) lipid nanoparticles (LRC) and with B) nanoparticles coated with HA (HLRC) at different N/P ratios (1, 3, 3.5, 5) after 24 h. C) Cell viability was evaluated by staining with propidium iodide (PI) and annexin V in order to view necrosis and apoptosis at specified times after transfection with either LRC or HLRC at an N/P ratio of 3. The internalization of LRC D) and HLRC E) by THP-1 cells was measured at specified times by flow cytometry, using rhodamine-fluorescent nanoparticles and with phase contrast and fluorescence microscopy 24 h post-transfection. All the values are means±standard deviation of the mean, and the statistical analyses were calculated using one-way ANOVA.

FIG. 4 shows the electrophoresis study of the formulations of mRNA complexed with DOPE/DOTAP/Coatsome SS-M liposomes at different N/P ratios (1, 5, 10 and 50) in the presence or absence of heparin in order to demonstrate complexation and the presence of the nucleic acid in the lipoplexes.

FIG. 5 reports the results in terms of transfection efficiency and cell viability of THP-1 cells after transfection with complexed LRC and HLRC (DOTAP/DOPE/Coatsome SS-M; microfluidic production), with eGFP-mRNA at different N/P ratios. Flow cytometry analysis of transfection efficiency and fluorescence intensity of THP-1 monocytes with A) LRC and B) HLRC at different N/P ratios (1, 2, 2.5, 3, 3.5, 5) after 24 h. C) Cell viability evaluated by staining with propidium iodide (PI) and annexin V in order to view necrosis and apoptosis at specified times after transfection with either LRC or HLRC at an N/P ratio of 2. All the values are means±standard deviation of the mean, and statistical analyses were calculated using one-way ANOVA.

FIG. 6 reports the transfection efficiency of different lipid formulations loaded with mRNA at N/P ratios of 1.5 and 3 on C2C12 myoblasts for inducing GFP expression.

FIG. 7 reports the results in terms of transfection efficiency of C2C12 myoblasts after transfection with complexed LRC and HLRC (DOTAP/DOPE/Coatsome SS-M; microfluidic production) complexed with mRNA at an N/P ratio of 1.5 for inducing GFP expression.

FIG. 8 shows the distribution of HLRC (DOTAP/DOPE/Coatsome SS-M; microfluidic production) labeled with PE-cyanine5 and associated with mRNA encoding the fluorescent protein mCherry after intramuscular administration into the tibialis anterior of healthy Balb/C mice.

FIG. 9 shows the physicochemical properties of HLRC (DOTAP/DOPE/Coatsome SS-M; microfluidic production) loaded with pDNA. Mean diameter and polydispersity index (A) and zeta potential values (B) of the HLRC loaded with pDNA at different N/P ratios (1.5, 2, 3 or 4). Electrophoretic analysis of the HLRC loaded with pDNA, for researching the efficiency of association of DNA in HLRC (C). CryoTEM image of HLRC loaded with pDNA at an N/P ratio of 1.5 (D).

FIG. 10 shows the degree of internalization of the HLRC (DOTAP/DOPE/Coatsome SS-M; microfluidic production) loaded with pDNA on C2C12 myoblasts.

FIG. 11 shows the transfection efficiency of the HLRC (DOTAP/DOPE/Coatsome SS-M; microfluidic production) loaded with pDNA at different N/P ratios on C2C12 myoblasts for inducing GFP expression.

FIG. 12 reports the transfection efficiency of different lipid formulations loaded with pDNA at N/P ratios of 1.5 and 3 on C2C12 myoblasts for inducing GFP expression.

FIG. 13 shows the result of the cell viability assays on C2C12 myoblasts transfected with HLRC (DOTAP/DOPE/Coatsome SS-M; microfluidic production) loaded with pDNA at different N/P ratios. The values represent means±SD (standard deviation).

FIG. 14 shows the transfection efficiency result of the siRNA-HLRC (DOTAP/DOPE/Coatsome SS-M; microfluidic production) at different doses on C2C12 myoblasts for inhibiting GFP expression.

FIG. 15 reports the transfection efficiency, on C2C12 myoblasts, of LRC and HLRC (DOTAP/DOPE/Coatsome SS-M; microfluidic production) complexed at an N/P ratio of 1.5 and 3 with pDNA (encoding the protein mScarlet) and mRNA (encoding GFP protein) at a weight ratio of 2:1. The transfection efficiency is evaluated on the ability of the system to induce expression of GFP, mScarlet and simultaneously both of these at the same time.

### DETAILED DESCRIPTION OF THE INVENTION

### Definitions

“Lipoplex” means a complex that vectorizes a nucleic acid, in particular a complex between (i) a nucleic acid and (ii) a nanoparticle as defined in the present description.

“Cationic lipid” means a lipid having a net positive charge. A cationic lipid comprises a cationic polar head and one or more hydrophobic chain(s).

“Neutral lipid” means a lipid having a net neutral charge, more particularly a zwitterionic lipid.

“Micelle” means a spheroidal aggregate of amphiphilic molecules having a hydrophilic polar head and a hydrophobic chain.

“Liposome” means an artificial vesicle formed by concentric lipid bilayers that trap aqueous compartments between them.

The “zeta potential” represents the electric charge that a particle acquires by virtue of the cloud of ions that surrounds it when it is in suspension or solution. Indeed, when said particle moves in a liquid, it is surrounded by ions organized into an “electrical double layer”:
some of the ions are attached to the particle, thereby forming a layer of adhering ions referred to as the Stem layer,
the other portion of the ions forms a non-bonded layer, referred to as the diffuse layer.

The “slipping plane” delimits these two layers. The potential difference between the dispersion medium and the potential at the slipping plane defines the zeta potential. This potential represents the measure of the intensity of electrostatic repulsion or attraction between particles. A positive ζ (zeta) potential is crucial for ensuring the efficiency of complexation of the nucleic acid, also having an impact on the colloidal stability of the particles due to the electrostatic repulsion between said particles.

### Cationic Lipids

The nanoparticles of the invention comprise at least one cationic lipid (a).

The cationic lipid is preferably selected from:
lipopolyamines, such as 2-{3-[bis(3-aminopropyl)amino]propylamino}-N-ditetradecylcarbamoyl methyl-acetamide (compound RPR209120), 2-{3-[3-(3-aminopropylamino)propylamino]propylamino}-N,N-dioctadecyl-acetamide (RPR120535) (Byk et al, J. Med. Chem., 41, 224-235, 1998) or 2,3-dioleoyloxy-N-[2(sperminecarboxamido)ethyl]-N,N-dimethyl-1-propanaminium-trifluoracetate (DOSPA), dioctadecylamine-glycine-spermine (DOGS), dipalmitylphosphatidylethanolamine-5-carboxyspermylamide (DPPES);
quaternary ammoniums, such as 1,2-dimyristoyloxypropyl-3-dimethylhydroxyethyl ammonium bromide (DMRIE), N-(2,3-dioleyloxypropyl]-N,N,N-trimethylammonium chloride (DOTMA), 1,2-dioleoyloxypropyl-N,N,N-trimethylammonium chloride (DOTAP), dimethyldioctadecylammonium bromide (DDAB), or 1,2-dioleyloxypropyl-3-dimethylhydroxyethylammonium bromide (DORIE), N-(4-carboxybenzyl)-N,N-dimethyl-2,3-bis(oleoyloxy)propan-1-aminium (DOBAQ), 1,2-dioleoyl-sn-glycero-3-ethylphosphocholine (chloride salt) (DOEPC), N—N-dioleoyl-N,N-dimethylammonium chloride (DODAC), 1,2-dioleoyl-3-dimethylammonium-propane (DODAP), N-methyl-4(dioleyl)methylpyridiniumchloride (SAINT-2);
stearylamine (SA), N-t-butyl-N′-tetradecyl-3-tetradecylaminopropionamidine (DiC14-amidine), O,O′-dimyristyl N-lysylaspartate (DMKD), or O,O′-dimyristyl-N-lysyl glutamate (DMKE); and
lipids including cationic heads of guanidine (BGTC) or imidazole (DOTIM) type,
   alone or as a mixture.

In a preferred embodiment, the cationic lipid is selected from quaternary ammoniums such as are mentioned above.

In a preferred embodiment, the cationic lipid is DOTAP.

Advantageously, the cationic lipid is present in the nanoparticle in an amount of between 3 mM and 15 mM, preferably between 5 and 10 mM, more preferably still 5 mM, and/or the mole ratio of cationic lipid to the other lipids is between 1:1 and 2:1.

In a preferred embodiment, the cationic lipid (a) is DOTAP and the mole ratio of DOTAP to all of the other lipids (b) and (c) is between 1:1 and 2:1, preferably approximately 1:1. DOTAP is preferably present in an amount of between 3 mM and 15 mM, preferably between 5 and 10 mM, more preferably still 5 mM.

### Neutral Lipids

The nanoparticles of the invention also comprise at least one neutral lipid (b).

Examples of neutral lipid include 1,2-dioleoyl-sn-glycero-3-phosphoethanolamine (DOPE), dioleoyl-sn-glycero-3-phosphocholine (DOPC), dipalmitoyl-sn-glycero-3-phosphocholine (DPPC) and N′-(rac-1-[1 1-(F-octyl)undec-10-enyl]-2-(hexadecyl)glycero-3-phosphoethanoyl)sperminecarboxamide), which can be used alone or as a mixture. The neutral lipid is preferably DOPE.

A preferred nanoparticle comprises a mixture of DOTAP and DOPE. The mixture of DOTAP and DOPE is preferably in the form of a liposome or lipid nanoparticle.

Advantageously, the nanoparticle of the invention is devoid of cholesterol, or even of any steroid.

Advantageously, the neutral lipid is present in the nanoparticle in an amount of between 1 mM and 10 mM, preferably between 1 and 5 mM, preferably approximately 2.5 mM.

pH-Sensitive Lipids

The nanoparticles of the invention also comprise at least one pH-sensitive lipid (c) which differs from the cationic lipid (a). These lipids are protonated at a low pH, which gives them a positive charge, but remain neutral at physiological pH.

“pH-sensitive lipid” means ionizable lipids, particularly lipids which react on the basis of variations in the pH of the surrounding medium and which are cleavable by acid hydrolysis, or which comprise reducible groups such as disulfide bridges.

These lipids are particularly liable to cleavage by a cellular reducing agent such as glutathione. The lipid typically contains two hydrophobic fractions and is functionalized with two sensitive units which can respond to the intracellular environment. The first is a tertiary amine, a unit which acquires a positive charge in response to an acid compartment (such as the endosome or lysosome) for destabilization of the membrane; the second is a disulfide bridge, a unit which can be cleaved in response to a reducing environment (such as in the cytoplasm) for spontaneous breakdown.

A preferred example of pH-sensitive lipid is thus known under the name ssPalm (for “SS-cleavable and pH-activated lipid-like material”).

The Coatsome® products sold by NOF America corporation are preferred pH-sensitive lipids of this kind. They are particularly described in the articles by Tanaka et al, Biomaterials, 2014, 35(5): 755-1761 and Hidetaka Akita, Biol. Pharm. Bull. 2020, 43 (11): 1617-1625, and also the patent applications EP 3 315 125 by Silence Therapeutics, WO 2020/142725 by Oncorus; WO 2021/123332 by Curevac.

In a preferred embodiment, the pH-sensitive lipid is a lipid of the following formula (I):

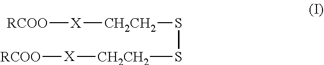

wherein:
RCOO is a group selected from the myristoyl group, the α-D-tocopherolsuccinoyl group, the linoleyl group and the oleoyl group; and
X is selected from the groups having the following structures (II), (III) or (IV):

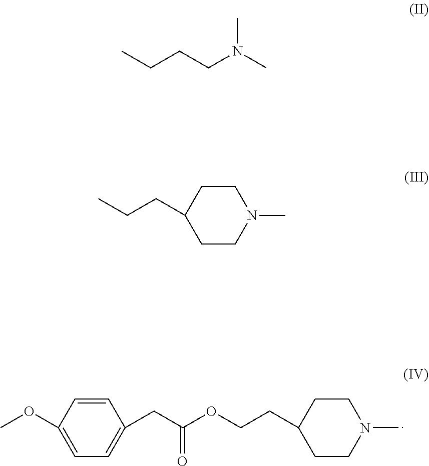

In particular, the pH-sensitive lipid is a lipid of formula (I) as defined above, wherein:
RCOO is a myristoyl group and X is a group of formula (II) (corresponding to the product Coatsome® SS-M [SS-14/3AP-01]);
RCOO is an α-D-tocopherolsuccinoyl group and X is a group of formula (II) or (III) (corresponding, respectively, to the product Coatsome® SS-E [SS-33/3AP-05] or Coatsome® SS-EC [SS-33/4PE-15]);
RCOO is a linoleyl or oleoyl group and X is a group of formula (III) (corresponding, respectively, to the product Coatsome® SS-LC [SS-18/4PE-13] or Coatsome® SS—OC [SS-18/4PE-16]); or
RCOO is an oleoyl group and X is a group of formula (IV) (corresponding to the product Coatsome® SS—OP).

Advantageously, the pH-sensitive lipid is a lipid of formula (I) as defined above, wherein —RCOO is a myristoyl group and X is a group of formula (II). This product is known as Coatsome® SS-M.

In another embodiment, the pH-sensitive lipid can be selected from the following lipids:
A lipid comprising a tertiary amine group, such as 1,2-dilinoleyloxy-n,n-dimethyl-3-aminopropane (DLinDMA), O—(Z,Z,Z,Z-heptatriaconta-6,9,26,29-tetraen-19-yl)-4-(N,N-dimethylamino) (DLin-MC3-DMA), or 2-[2,2-bis[(9Z,12Z)-octadeca-9,12-dienyl]-1,3-dioxolan-4-yl]-N,N-dimethylethanamine (DLin-KC2-DMA).

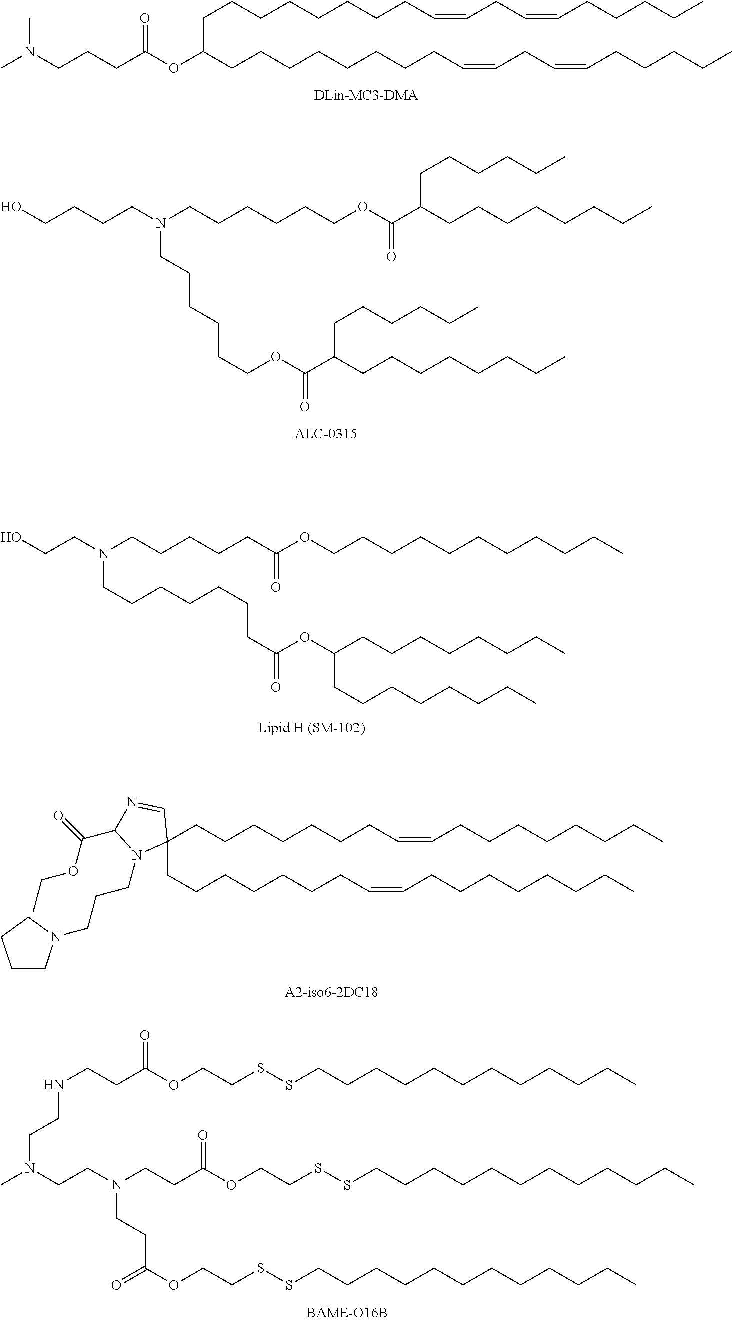

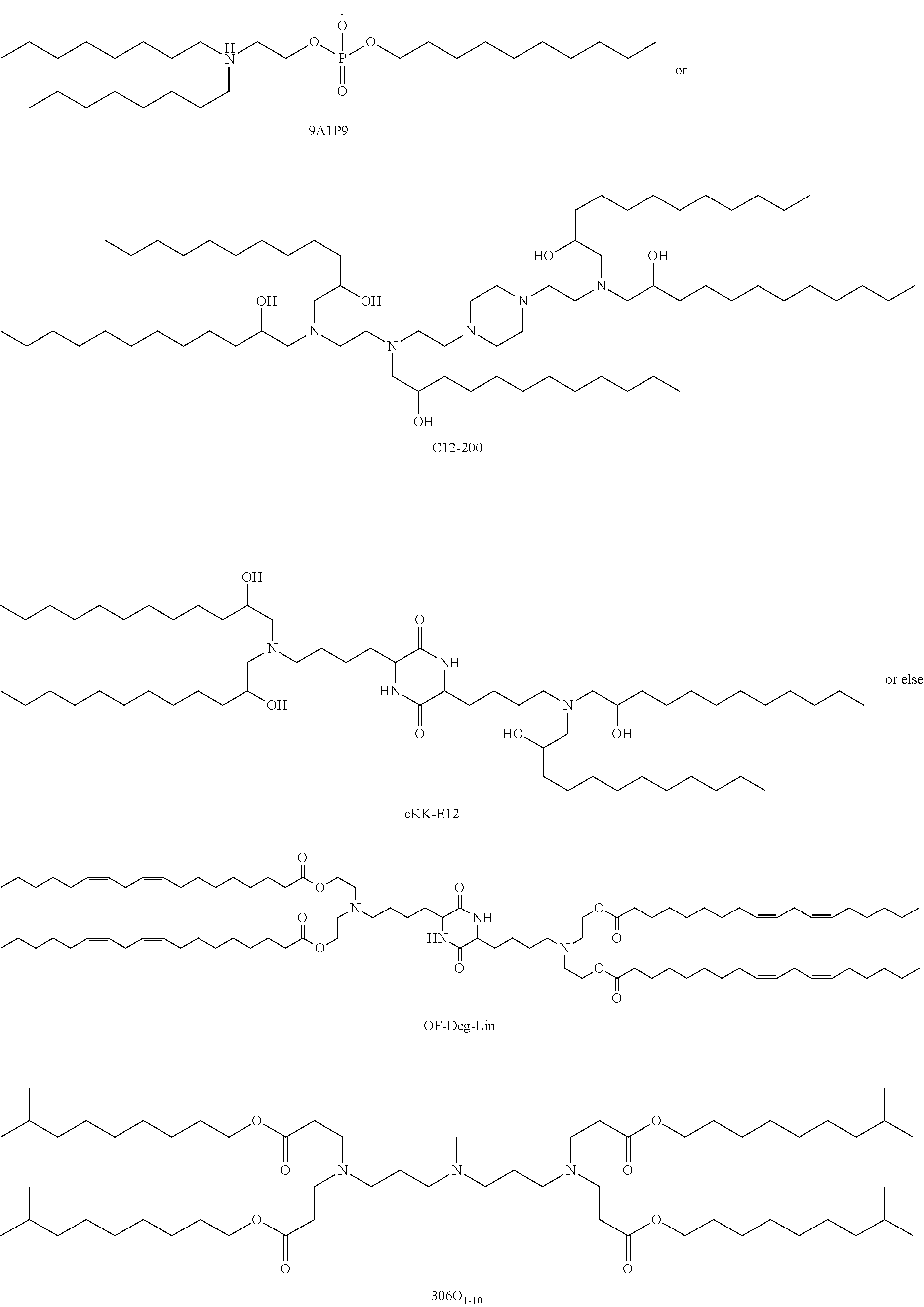

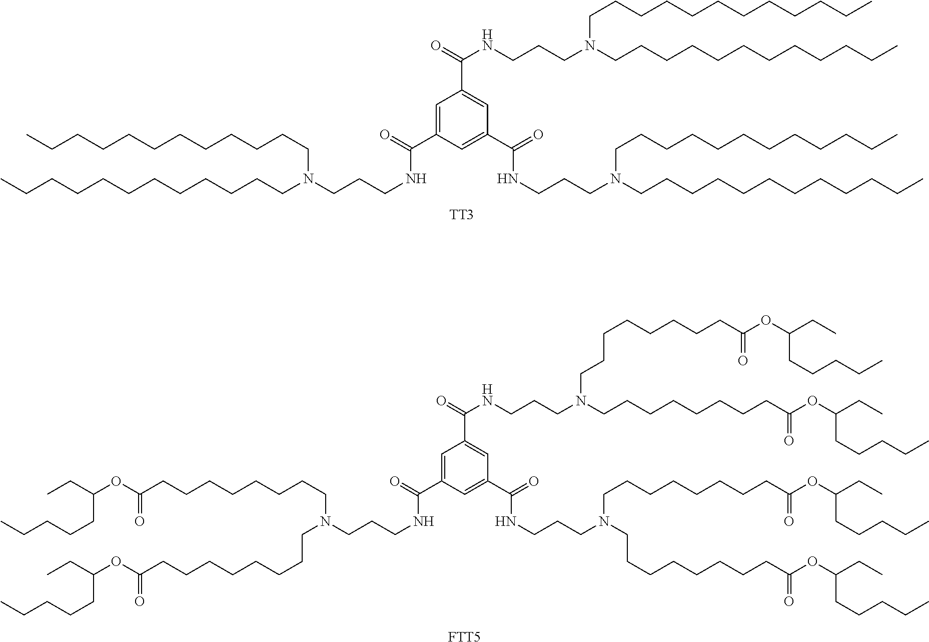

The pH-sensitive lipids mentioned above may be used alone or as a mixture with other pH-sensitive lipids.

Advantageously, the pH-sensitive lipid is present in the nanoparticle in an amount of between 1 and 10 mM, preferably between 1 and 5 mM, more preferably still approximately 2.5 mM. In a preferred embodiment, the amount of pH-sensitive lipid is practically identical to the amount of neutral lipid.

Advantageously, the pH-sensitive lipid is a lipid of formula (I) as defined above, wherein —RCOO is a myristoyl group and X is a group of formula (II), in an amount of between 1 and 10 mM, preferably between 1 and 5 mM, more preferably still approximately 2.5 mM.

A preferred nanoparticle comprises a mixture of
a) DOTAP as cationic lipid,
b) DOPE as neutral lipid, and
c) a lipid of formula I as defined above, as pH-sensitive lipid, preferably a lipid of formula (I) wherein —RCOO is a myristoyl group and X is a group of formula (II).

A particularly preferred nanoparticle comprises a mixture of DOTAP (in an amount of 5 mM), DOPE (in an amount of 2.5 mM) and a lipid of formula (I) as defined above, wherein —RCOO is a myristoyl group and X is a group of formula (II), in an amount of 2.5 mM.

In another preferred embodiment, the nanoparticle comprises
(a) DOTAP as cationic lipid,
(b) DOPE as neutral lipid, and
(c) DLin-MC3-DMA as pH-sensitive lipid.

### Method for Preparing the Nanoparticle

The nanoparticle of the invention is preferably prepared in the form of a liposome or a lipid nanoparticle comprising a) a cationic lipid, b) a neutral lipid and c) a pH-sensitive lipid which differs from the cationic lipid (a), in a mole ratio of 2:1:1 to 4:1:1.

Advantageously, the nanoparticle according to the invention is prepared in the form of a liposome comprising:
(a) approximately 50% of a cationic lipid,
(b) approximately 25% of a neutral lipid, and
(c) approximately 25% of a pH-sensitive lipid which differs from the cationic lipid (a).

According to a preferred aspect of the invention, the nanoparticle according to the invention is prepared in the form of a liposome or a lipid nanoparticle comprising:
(a) approximately 50% of DOTAP as cationic lipid,
(b) approximately 25% of DOPE as neutral lipid, and
(c) approximately 25% of a pH-sensitive lipid selected from the lipids of formula (I) as defined above, wherein:
RCOO is a group selected from the myristoyl group, the α-D-tocopherolsuccinoyl group, the linoleyl group and the oleoyl group; and
X is selected from the groups having the structures (II), (III) or (IV) as defined above.

According to a preferred aspect of the invention, the nanoparticle according to the invention is prepared in the form of a liposome or a lipid nanoparticle comprising:
(a) approximately 50% of DOTAP as cationic lipid,
(b) approximately 25% of DOPE as neutral lipid, and
(c) approximately 25% of a pH-sensitive lipid selected from the lipids of formula (I) as defined above, wherein:
   RCOO is a myristoyl group and X is a group of formula (II);
   RCOO is an α-D-tocopherolsuccinoyl group and X is a group of formula (II) or (III);
   RCOO is a linoleyl or oleoyl group and X is a group of formula (III); or
   RCOO is an oleoyl group and X is a group of formula (IV).

The liposomes or lipid nanoparticle can be obtained using techniques known to those skilled in the art, particularly by a method of hydrating the lipid film and extrusion (typically through one or more membranes, for example polycarbonate membranes) or by a microfluidic process. These two methods are described in detail in the examples below.

Complexation with the Nucleic Acid

According to a particular aspect of the invention, the nanoparticle is loaded with a nucleic acid, particularly for forming lipoplexes. Lipoplexes are in particular formed by complexation of lipids with a nucleic acid. In the complex obtained, the lipids and the nucleic acid are non-covalently associated.

The loaded nanoparticle, or lipoplex, can particularly be obtained by a process comprising:
i) obtaining a mixture of lipids a), b) and c) as defined above (which makes it possible to obtain liposomes), and
ii) bringing said mixture into contact with a nucleic acid, to obtain complexes or lipoplexes.

Step i) of this process is preferably carried out according to the method described above for forming the lipid nanoparticle.

Step ii) is preferably carried out by diluting the liposomes obtained in step i) in a buffer or aqueous solvent, particularly in water devoid of RNase, then by adding a solution of nucleic acids in the same buffer or the same aqueous solvent, particularly in water devoice of RNase.

According to a particular aspect of the invention, the nanoparticle may be loaded with the nucleic acid so as to obtain an N/P ratio ranging from 1 to 100, preferably 1 to 60, preferably 1 to 50, preferably 1 to 40, preferably 1 to 30, more preferably still 1 to 20, preferably 1 to 10, preferably 1 to 8, more preferably still 1.5 to 8, preferably 1 to 5, more preferably still 1.5 to 5 or 1.5 to 4.

The N/P charge ratio corresponds to the ratio of the number of amine functions of the lipids of the nanoparticle (positive charges) to the number of phosphate molecules of the nucleic acid (negative charges).

According to a specific aspect of the invention, when the loaded nanoparticle is formed by a process of hydrating the lipid film and extrusion, the loaded nanoparticle, or lipoplex, typically has an N/P ratio of greater than or equal to 3, preferably 3 to 8, preferably 3 to 5.

According to another aspect, when the loaded nanoparticle is formed by a microfluidic process, the loaded nanoparticle, or lipoplex, typically has an N/P ratio of greater than or equal to 1.5 or 2, preferably 1.5 to 10, preferably 1.5 to 5 or 2 to 5.

The nucleic acid can be a deoxyribonucleic acid (DNA), a single-stranded or double-stranded ribonucleic acid (RNA), for example an mRNA or interfering RNA, or a mixture, or hybrid DNA/RNA sequences. These may be sequences of natural or artificial origin. They may also be obtained by chemical modification of the sugar parts thereof, the nucleobase parts thereof or the internucleotide backbone thereof. Among the advantageous modifications in the sugar parts, mention may be made of modifications at the 2′ position of ribose, such as 2′-deoxy, 2′-fluoro, 2-amino, 2′-thio, or 2′-O-alkyl modifications, in particular 2′-O-methyl modifications, instead of the usual 2′-OH group on ribonucleotides, or else the presence of a methylene bridge between the 2′ and 4′ positions of ribose (LNA). Regarding the nucleobases, use may be made of modified bases, such as in particular 5-bromouridine, 5-iodouridine, N<3>-methyluridine, a 2,6-diaminopurine (DAP), 5-methyl-2′-deoxycytidine, 5-(1-propynyl)-2′-deoxyuridine (pdU), 5-(1-propynyl)-2′-deoxycytidine (pdC), or bases conjugated to cholesterol. Finally, advantageous modifications to the internucleotide backbone include replacing phosphodiester groups of this backbone with phosphorothioate, methylphosphonate, phosphorodiamidate groups, or using a backbone composed of N-(2-aminoethyl)glycine units bonded by peptide bonds (PNA, peptide nucleic acid). Of course, the various modifications (base, sugar, backbone) can be combined to give modified nucleic acids of morpholino type (bases attached to a morpholine ring and bonded by phosphorodiamidate groups) or PNA type (bases attached to N-(2-aminoethyl)glycine units bonded by peptide bonds).

The nucleic acids may be small (for example smaller than 200 nucleotides), such as antisense oligonucleotides, interfering RNAs (miRNA, siRNA, etc.), or aptamers, for example. They may also advantageously be large (for example greater than 200, 300 or 400 nucleotides). In a particular embodiment, the nucleic acid is a DNA plasmid (pDNA).

Examples of sizes of nucleic acids which can be loaded into the lipoplexes of the invention are presented in table 1 below.

TABLE 1

| Nucleic acid | Molecular weight (kDa) | Nucleotides |
|---|---|---|
| pDNA | Between 3000 and 5000, | Between 4000 and 8000 bp, |
| | for example approximately | preferably approximately |
| | 4000 | 6000 |
| mRNA | Between 200 and 600, for | Between 400 and 2000, for |
| | example approximately 400 | example approximately 1000 |
| | | or 1200 |
| siRNA | Between 5 and 20, for | Between 10 and 30, |
| | example 12 | preferably approximately 20 |
| | | (bp) |
| Antisense | Between 1 and 10, for | Between 8 and 20, preferably |
| oligonucle- | example 5 | approximately 14 |
| otide (ASO) | | |

In a preferred embodiment, the nucleic acid is an mRNA.

In another embodiment, the nucleic acid is a DNA.

In another embodiment, the nucleic acid is an RNA other than mRNA, for example an interfering RNA (for example siRNA or miRNA).

In a preferred embodiment, the nanoparticle comprises a DOPE/DOTAP and SS-Palm mixture, charged with an RNA (such as an mRNA or an interfering RNA) or a DNA, preferably a pDNA.

In another preferred embodiment, the nanoparticle comprises a DOPE/DOTAP and SS-Palm mixture, loaded with a mixture of RNA and DNA, preferably a mixture of mRNA and pDNA, for example at an RNA:DNA ratio ranging from 1:1 to 1:10, preferably from 1:1 to 1:5, preferably from 1:1 to 1:3, more preferably still 1:2.

### Anionic Polysaccharide

The nanoparticle may be coated with molecules of one or more anionic polysaccharides.

A “polysaccharide” is formed by sequences of saccharides interconnected by glycosidic linkages, and an “anionic polysaccharide” is a polysaccharide having a net negative charge. Among the anionic polysaccharides, mention may be made of glycosaminoglycans (such as hyaluronic acid or a salt thereof, alginic acid or a salt thereof), carrageenans (sulfated red algae polysaccharides), fucans (sulfated brown algae polysaccharides), carboxymethyl benzylamide sulfonate dextrans or CMDBS (synthetic polysaccharides prepared from dextran by statistical substitution of hydroxyl functions with carboxymethyl, benzylamide, sulfonate and sulfate chemical functions), and heparan sulfates (complex polysaccharides belonging to the glycosaminoglycan family).

In a preferred embodiment, the anionic polysaccharide is hyaluronic acid or a salt thereof, such as the sodium salt.

Alternatively, the anionic polysaccharide may be alginic acid or a salt thereof, such as the sodium salt.

In a preferred embodiment, the nanoparticle comprises a mixture of DOPE/DOTAP and an SS palm lipid of formula I, is loaded with an RNA (for example of the mRNA or siRNA type) or a DNA, preferably a pDNA, and is coated with molecules of hyaluronic acid or a salt thereof, such as sodium hyaluronate (HA).

The coating with the anionic polysaccharide is preferably achieved by bringing the nanoparticle/liposomes/lipoplexes previously obtained into contact with the anionic polysaccharide such as hyaluronic acid in an aqueous solvent, for example in water devoid of RNase.

The lipoplexes are preferably coated with anionic polysaccharide, such as HA, at a cationic lipid:anionic polysaccharide weight ratio of 1:2.

Covering the lipoplexes with anionic polysaccharides has the particular advantage of increasing the accumulation of the lipoplexes at tumor sites, or at cells, for instance macrophages, myoblasts or myotubes, where cell receptors for these polysaccharides (such as CD44) are overexpressed.

### Transfer of Nucleic Acids

The nanoparticles or lipoplexes of the invention can be used for the transfer of nucleic acids into cells in vivo, in vitro or ex vivo. In particular, the compositions according to the invention can be used to highly effectively transfer nucleic acids into numerous cell types.

The nanoparticle according to the present invention is preferably present in a medium containing the cells to be transfected, under conditions such that the lipoplex passes from the medium into the cytoplasm of the cells, then the nucleic acid is released in the cytosol and/or nucleus of the cells.

In a particular embodiment, the nanoparticle may also comprise a targeting element that makes it possible to direct the transfer of the nucleic acid, such as an intracellular (nucleus, etc.) targeting element and/or extracellular targeting element (targeting of certain cell/tissue types). Another subject of the present invention is a pharmaceutical composition comprising a nanoparticle or lipoplex as defined herein and a pharmaceutically acceptable carrier.

Advantageously, the aim of the invention is the use of a nanoparticle as described herein as a pharmaceutically acceptable vector of a nucleic acid.

The doses of nucleic acid used, and the number of administrations, can be adapted based on different parameters, and particularly based on the mode of administration used, on the disease in question, on the nucleic acid to be administered, or else on the desired duration of the treatment.

In a preferred embodiment, the nanoparticles of the invention are of use in gene therapy applications. The nucleic acid can then preferably be a DNA encoding a functional protein, particularly a protein which is not produced functionally in the patient.

In a plurality of embodiments, the nucleic acid may be an mRNA encoding a protein which is not produced functionally in the patient, or a protein of therapeutic or vaccine-related interest. According to another aspect, the nucleic acid selected may inhibit the expression of a protein expressed by the subject, and thus be an iRNA or an antisense oligonucleotide, for example, which is complementary to the mRNA sequence it is targeting. It can thus modify the mRNA, particularly by exon skipping or inclusion by acting on the splicing stage.

In other embodiments, the nanoparticles of the invention are of use in ex vivo applications, for example to modify cells, particularly with the aim of cell therapy.

A particular therapeutic application relates to treating muscle disorders or musculoskeletal disorders.

A method for releasing a nucleic acid into the muscle of a subject, preferably a human patient suffering from a muscle or musculoskeletal disorder, by intramuscular administration of a nanoparticle as described herein, loaded with a nucleic acid, for example an mRNA encoding a protein of therapeutic interest, is also described herein.

The examples and figures illustrate the invention without limiting the scope thereof.

### Example 1: Production of Liposomes (Lipoplexes) as Carriers for Intracellular mRNA Administration

### Materials and Methods

### Materials

The cationic lipid 1,2-dioleoyl-3-trimethylammonium propane (DOTAP) and auxiliary lipid 1,2-dioleoyl-sn-glycero-3-phosphoethanolamine (DOPE) were purchased from Avanti Polar Lipids. Coatsome® SS-M was obtained from NOF America Corporation. The mRNA encoding GFP and Viromer were supplied by BioNTech. The sodium hyaluronate (HA, weight-average molar mass, Mw=20 000 g·mol⁻¹) was purchased from Lifecore Biomedical (Minnesota, USA). The Live/Dead® fixable was supplied by Sigma-Aldrich (Saint-Quentin-Fallavier, France).

### Formulation of Liposomes and Lipoplexes

Formulation of liposomes The liposomes were generated using the thin-film method, followed by extrusion. The lipids were dissolved in chloroform to generate solutions at the final desired concentration, and then were added to a 10 mL round-bottomed flask for a final lipid concentration of 10 mM after hydration, following the mole ratio indicated in table 2. The organic solvent was eliminated under reduced pressure at 40° C. using a rotary evaporator. To completely remove the chloroform, the round-bottomed flasks were left overnight in a dryer. The lipid film was rehydrated using HEPES buffer 0.1 M, pH 5.5. After 5 minutes of vortexing, the multilamellar vesicles were extruded through a large polycarbonate membrane (400 nm) using an Avanti mini extruder (Avanti Polar Lipids), followed by a second extrusion with a 100 nm membrane in order to obtain unilamellar vesicles. Fluorescent liposomes were prepared with rhodamine-labeled DOPE (Rho-PE) embedded in the liposome membrane. Rho-PE was added at a ratio of 2% w/w to the lipid solution before the drying process. DTPA liposomes were prepared with DSPE-DTPA (PE-DTPA) for radiolabeling purposes. 0.5% mol/mol of PE-DTPA was added to the lipid solution before the drying process.

TABLE 2
Lipid composition

| | | | | Conc. |
|---|---|---|---|---|
| | Final conc. | Conc. DOTAP | Conc. DOPE | Coatsome ® |
| | lipids (mM) | (mM) | (mM) | (mM) |
| Liposome | 10 | 5 | 2.5 | 2.5 |

Formulation of lipoplexes The lipoplexes were prepared by diluting the liposomes at 50 μL with water devoid of RNase to a final concentration of 1.8 mM, and by then adding 50 μL of different concentrations of mRNA (0.03-0.15 mg/mL) in water devoid of RNAse to obtain complexes in the N/P ratio range from 1 to 5, as shown in table 3. Lipoplexes coated with hyaluronic acid were prepared by adding 50 μL of a solution of HA at 0.6 mg/mL in water devoid of RNase to the already formulated liposomes. Intended for in vivo purposes, the lipoplexes were diluted with a 10% glucose solution or coated with HA dissolved in 10% glucose in order to obtain the same final concentration and good osmolality.

TABLE 3
Mole ratios

| | Weight of | Weight of | | |
|---|---|---|---|---|
| N/P ratio | lipid (g) | mRNA (g) | N+ (mol) | P− (mol) |
| 1.0 | 6.43E−05 | 1.53E−05 | 4.50E−08 | 4.51E−08 |
| 1.5 | 6.43E−05 | 1.02E−05 | 4.50E−08 | 3.00E−08 |
| 2.0 | 6.43E−05 | 0.77E−05 | 4.50E−08 | 2.25E−08 |
| 2.5 | 6.43E−05 | 0.61E−05 | 4.50E−08 | 1.80E−08 |
| 3.0 | 6.43E−05 | 0.51E−05 | 4.50E−08 | 1.50E−08 |
| 3.5 | 6.43E−05 | 0.44E−05 | 4.50E−08 | 1.28E−08 |
| 5 | 6.43E−05 | 0.31E−05 | 4.50E−08 | 0.90E−08 |

### Physicochemical Analysis of the Lipoplexes

The particle size distribution and the surface electrical charge were measured using the Nano ZS Zetasizer® from Malvern (Malvern Instruments S.A, Worcestershire, UK). The hydrodynamic diameter and the polydispersity index (PDI) were measured by dynamic light scattering (DLS) using the cumulant method, these measurements being taken at 25° C. at a 173° detection angle. The zeta (ζ) potential was determined by the electrophoresis technique. All the samples were diluted in 1 mM of NaCl and the analyses were performed in triplicate.

The efficiency of mRNA association was determined using Sybr Green® agarose gel electrophoresis. Samples were diluted in HEPES at pH 5.5 to a final amount of RNA of 0.125 μg. After adding mRNA gel loading stain, the samples were tested on 1% (w/v) agarose gel for 30 min at 80 V. The mRNA bands were visualized and imaged using the EZ imaging device from Gel Doc™ (Bio-Rad).

Analysis by Transmission Electron Cryomicroscopy (cryoTEM) of the Liposomes Loaded with mRNA

The morphology of the lipid nanoparticles was evaluated using transmission electron cryomicroscopy. In summary, 3.5 μL of formulation were added to a CF-2/1-3Cu-50 copper mesh and frozen by submersion using a Vitrobot (Thermoscientific) to generate vitreous ice. The samples were stored in liquid nitrogen and imaged using a JEM-1400Flash electron microscope operating at 120 kV.

### Cell Studies on THP-1

Transfection study A reverse transfection method was used to amplify and prolong gene expression. Freshly prepared lipoplexes at an N/P ratio of 1, 3, 3.5 and 5 were placed in 24-well plates. THP-1 cells were added to the lipoplexes at a density of 150 000 cells in a total of 600 μL of complete RPMI medium supplemented with 15% FBS, 1 mM sodium pyruvate, 10% HEPES buffer, 1% L-glutamine and 1% penicillin/streptomycin. The lipoplex volumes were adapted in order to transfect cells with 3 μg of eGFP mRNA for each condition. A solution of Viromer Red containing eGFP mRNA was used as positive control for the transfection; 3 μg of e-GFP mRNA were placed in plates. The cells were subsequently incubated for 24 h at 37° C. The cell medium was modified after 4 h of transfection. After transfection, the cells were washed and stained with a solution of Fixable Live/Dead Violet before taking readings using a BD LSR 2 flow cytometer. The data was analyzed using the FlowJo software and statistical analysis was determined using the GraphPad Prism v8.1 software.

Viability studies THP-1 cells and the lipoplexes were plated as described above. The lipoplexes were freshly prepared and added to each well in the necessary amount to have 3 μg of eGFP mRNA. Treatment with 50 μM cisplatin was used as positive control for cell death. The cells were subsequently incubated for 4 h, 24 h, and 48 h at 37° C., 5% CO2. The cell medium was replaced after 4 h of transfection. The cells were subsequently washed and stained with annexin V Pacific Blue™ and diluted at 1:50 propidium iodide before taking readings using a BD LSR 2 flow cytometer. The data was analyzed using the FlowJo software and statistical analysis was carried out using the GraphPad Prism v8.1 software.

Internalization study Freshly prepared lipoplexes of liposomes associated with PE-Rho at an N/P ratio of 3 were plated into 24-well plates. The THP-1 cells were subsequently added at a density of 150 000 cells in a total of 600 μL of complete RPMI medium. The lipoplex volumes were adapted in order to transfect cells with 3 μg of eGFP mRNA for each condition. The cells were incubated for 2, 4, 24 and 48 h at 37° C. The cell medium was changed after 4 h of transfection. After transfection, the cells were washed and stained with a Fixable Live/Dead Violet solution before taking readings using a BD™LSR II flow cytometer. The data was analyzed using the FlowJo software and statistical analysis was determined using the GraphPad Prism v8.1 software.

### In Vivo Biodistribution Study

In vivo biodistribution of radiolabeled liposomes. The surface of the liposomes was radiolabeled by incubating 4.17 μmol (500 μL) of liposome (DOTAP-DOPE-Coatsome-SS-M) with 85.8 MBq (100 μL) of 111In at 60° C. for 30 minutes. This leads to an amount of radioactivity of 20.58 GBq/mmol. The molar/activity ratio was 0.24 nmol of PE-DTPA/MBq. Radiolabeling efficiency was evaluated by instant thin layer chromatography on silica gel strips (iTLC-SG, Biodex Medical Systems, Shirley, USA) using 100 mM citrate buffer, pH 5, as mobile phase. Free and labeled ¹¹¹In activity were assessed in an ionization chamber (Capintec, Florham Park, USA). Lipoplexes were prepared at an N/P ratio of 3. Briefly, 1.25 μmol (150 μL) of radiolabeled liposomes (0.63 μmol of DOTAP) were incubated at ambient temperature with 0.2 nmol mRNA respectively (equivalent to 0.244 μmol of phosphate), for 30 minutes. mRNA solutions were prepared and quantified using a NanoDrop™ (Ozyme, Saint-Cyr-l'Ecole, France). Subsequently, a solution of 10% glucose or 0.025 μmol of hyaluronic acid dissolved in 10% glucose was also added to the lipoplexes to adjust osmolality.

Formulations were injected intravenously into BALB/c_Rj mice anesthetized with 3% isoflurane. The mice received an injection of 2.07±0.82 MBq ¹¹¹In chelated to DTPA lipoplexes, either as positively-charged lipoplexes LRC or as negatively-charged lipoplexes HLRC (specific activity of 20.58 and 25.77 Gbq/mmol of total lipid; 50 nmol of total lipid/mouse in 100 μL, n=18). For each formulation, groups of 3 mice were euthanized 6 h, 24 h and 48 h post-injection by cervical dislocation. Thereafter, the mice were dissected and the organs were collected in order to perform quantitative analysis of the cumulative activity. Ex vivo quantification of the radioactivity of the organ was performed using a Wizard 3″ gamma ray counter (Perkin Elmer, Waltham USA). For each formulation, one mouse of the 48 h group was imaged in the prone position at 6 h, 24 h and 48 h. All living animal acquisitions were performed with a NanoSPECT/CT™ in vivo animal imager (Bioscan Inc., Washington D C, USA). During acquisition, the animals were anesthetized with 1.5% isoflurane and breathing was monitored with a Model 1025 T small animal monitoring and gating system (SA Instruments Inc., Stony Brook, USA). Each acquisition was performed with a preclinical SPECT/CT multiplexing with multiplexing multi-pinhole apertures. The SPECT device acquired 24 of 256×256 pixels for every 15°. The scan duration was 100 seconds per projection. The reconstruction was performed with the manufacturer's software, HiSPECT, using an ordered subsets expectation maximization (OSEM) algorithm with 9 iterations and 4 subsets with an image voxel size of 0.6 mm.

### Results

### Characterization of the Lipoplexes

Preparation and complexation of the liposomes with IVT mRNA. Cationic liposomes (LP) were prepared using a lipid film hydration method followed by extrusion through a 100 nm MWCO membrane. The liposomal hydrodynamic diameter measured by DLS was 140.8±3.4 nm, with a PDI of less than 0.2. The stability of the formulation, assessed over a period of 4 months, indicated that the size and PDI remained unchanged for the entire duration of the experiments. Similar results were obtained with rhodamine-labeled liposomes (Rho-LP) and DTPA liposomes (DTPA-LP), the hydrodynamic size of which was 151±2.3 and 139.1±2.9 nm, respectively, with a PDI of less than 0.2 in both cases. PDI has a significant impact on the stability and bioavailability of the liposomes. In order to be stable, safe and effective, the liposome preparation must be homogeneous. An acceptable liposome formulation for administering medicaments should have a PDI value of less than 0.3. The surface charge of all the liposome formulations prepared was positive, in the range of +40-55 mV (table 4).

TABLE 4
Physicochemical characterization of LPs

| | LP | Rho-LP | DTPA-LP |
|---|---|---|---|
| Mean Z (nm) | 140.8 ± 3.4 | 151 ± 2.3 | 139.1 ± 2.9 |
| PDI | 0.09 ± 0.02 | 0.07 ± 0.02 | 0.13 ± 0.01 |
| ζ potential (mV) | +42.0 ± 2.4 | +49.5 ± 1.4 | +55.8 ± 1.8 |

The amount of positive charge present in the formulation is of critical importance when the electrostatic complexation occurs with a precise amount of negative charges. This is why a method for quantifying the charges borne by the lipids was developed using RP-HPLC. The final formulations showed approximately 95% content of DOTAP and DOPE, while the yield was lower in the case of SS-Palm (approximately 60%). This can be attributed to the pH of the formulation, which may cause partial protonation of the tertiary amine, leading to precipitation of part of the lipid. Two different types of complex were prepared (FIG. 1). In order to obtain liposome RNA complexes (LRC), the mRNA was mixed with liposomes at an N/P ratio ranging from 1 to 5. Seven different N/P ratios (1, 1.5, 2, 2.5, 3, 3.5 and 5) were tested in terms of physicochemical characteristics and ability to retain nucleic acids. With the aim of obtaining systems that have similar characteristics in terms of size but opposing characteristics in terms of surface charges, the same set of lipoplexes was coated with an anionic polymer, hyaluronic acid (HA), to form hybrid liposome-RNA complexes (HLRC). At physiological pH and at the pH used for liposome formulation (5.5), the phosphate groups mainly exist in their anionic form, enabling good complexation with the cationic lipid particle. The amount of positive charges in the lipids was determined based on information provided by the manufacturer. Seven different N/P ratios in the range between 1 and 5 were tested in terms of physicochemical characteristics and ability to retain nucleic acids.

TABLE 5
Characterization of LRCs

| N/P ratio | 1 | 1.5 | 2 | 2.5 | 3 | 3.5 | 5 |
|---|---|---|---|---|---|---|---|
| Mean Z | 213.0 ± | 2109.6 ± | 1287.24 ± | 605.35 ± | 257.04 ± | 241.33 ± | 207.8 ± |
| (nm) | 90.54 | 1425.91 | 778.80 | 389.31 | 102.70 | 103.74 | 88.36 |
| PDI | 0.16 ± | 0.40 ± | 0.50 ± | 0.37 ± | 0.17 ± | 0.17 ± | 0.17 ± |
| | 0.06 | 0.40 | 0.26 | 0.11 | 0.09 | 0.04 | 0.03 |
| ζ | −24.8 ± | +25.3 ± | +32.11 ± | +34.0 ± | +34.83 ± | +33.29 ± | +39.6 ± |
| potential | 9.95 | 9.00 | 9.25 | 6.2 | 5.72 | 5.13 | 8.4 |
| (mV) | | | | | | | |

TABLE 6
Characterization of HLRCs

| N/P ratio | 1 | 1.5 | 2 | 2.5 | 3 | 3.5 | 5 |
|---|---|---|---|---|---|---|---|
| Mean Z | 209.4 ± | 1081.65 ± | 1930.11 ± | 543.63 ± | 255.29 ± | 240.08 ± | 227.63 ± |
| (nm) | 36.41 | 254.36 | 635.71 | 237.44 | 21.46 | 14.72 | 8.66 |
| PDI | 0.18 ± | 0.54 ± | 0.37 ± | 0.43 ± | 0.17 ± | 0.19 ± | 0.17 ± |
| | 0.11 | 0.27 | 0.18 | 0.04 | 0.09 | 0.04 | 0.04 |
| ζ | −31.16 ± | −28.87 ± | −25.63 ± | −27.13 ± | −26.37 ± | −25.25 ± | −28.21 ± |
| potential | 13.21 | 8.77 | 7.29 | 5.92 | 3.89 | 4.22 | 2.04 |
| (mV) | | | | | | | |

For both LRC and H-LRC, the N/P ratio of between 1.5 and 2.5 showed the greatest instability in terms of complex formation, as can be observed in terms of the mean size and polydispersity values reported in tables 5 and 6. On the contrary, for an N/P ratio of equal to 1 and greater than 3, the sizes became smaller than 300 nm, with a good PDI of around 0.2 which is important for the stability and bioavailability of the system. The surface charges were positive for all the N/P ratios in the case of LRC, with the exception of the N/P ratio of 1, where the presence of an excess of mRNA relative to the amount of lipids leads to negatively-charged particles. Interestingly, LRC with an N/P ratio of 1 proved stable after preparation with an initially negative (potential, drifting toward positive values after 24 h, probably because of the formation of aggregates due to the instability of the system. For all the other N/P ratios, the surface charges were similar to those of the liposomes alone, with a reduction following the addition of mRNA. This could be the result of the electrostatic interaction between the cationic lipid and the negatively-charged backbone of the nucleic acid. On the other hand, the zeta potential of the HLRCs was negative for all the N/P ratios tested, because of the presence of hyaluronic acid: in fact, at neutral pH, the pKₐ of the carboxylic groups of the polymer is approximately 3-4. We used this coating to obtain lipoplexes with negative charges, with the aim of observing their different behavior in vitro and in vivo.

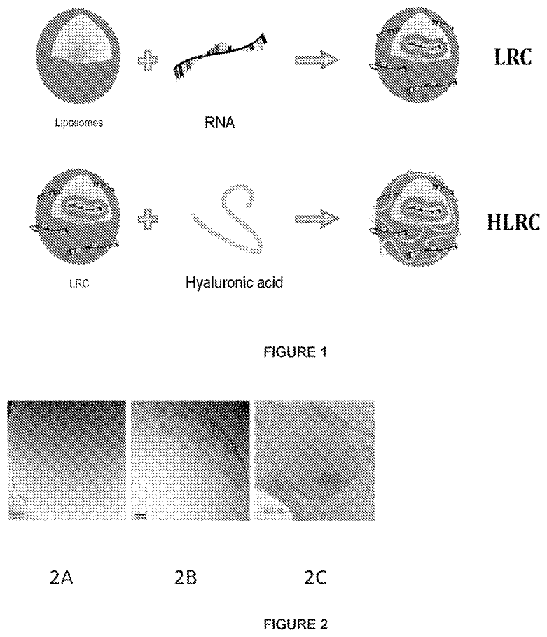

CRYOTEM. The LRCs and HLRCs do not consist of an ordered nucleic acid phase surrounded by an outer lipid bilayer; rather, they are partially condensed nucleic acid complexes with an ordered substructure and an irregular morphology. CryoTEM technology is a method for detecting single particles, used to characterize the morphology of the liposomes. FIG. 2A shows the conventional morphology of unilamellar liposomes. These are spherical vesicles having an aqueous internal compartment delimited by a single bilayer membrane. The interactions of the liposomes with mRNA and with mRNA/HA are shown in FIGS. 2B and 2C, respectively. The deformation and rearrangement of liposomal membranes result from strong electrostatic interactions between the cationic lipid head groups and the nucleic acid phosphate groups. Liposomes absorbing one another to form paired membranes, by virtue of the presence of mRNA which acts as a bridge between them, can be regularly observed. In fact, the contact plane between the bilayers had higher contrast than at their outer edges, suggesting electron-dense nucleic acid molecules sandwiched between the membranes. In other cases, as has already been reported in the literature for cationic liposomes and DNA, multilamellar systems form in which alternating nucleic acids and liposomes resemble a sandwich-type structure. It is said that this structural modification is beneficial for the stability of the nucleic acid: the bilayer packing promotes the protection of the mRNA molecules from degradation better than a simple surface association in which they would be more susceptible to degradation by serum nucleases.

Studies and stability of mRNA-liposome association. In order to evaluate the stability of the complexation of LRCs and HLRCs, electrophoretic assays were performed at all the N/P ratios studied. The nucleic acids migrate through an agarose gel matrix under the action of an electric field depending on their charge, size and morphology. In the case of the LRCs, a weak signal can be seen corresponding to free mRNA, meaning that a portion of the nucleic acid is not constantly bound to the cationic particle. Furthermore, the presence of the hyaluronic acid coating disturbs this release, and no fluorescence can be observed.

### In Vitro Cell Studies on THP-1

Transfection studies. In order to determine whether, and to what extent, the LRCs and HLRCs were capable of transfecting cells, we incubated them with THP-1 cells after complexation with IVT mRNA capable of inducing eGFP expression. The protocol was optimized, focusing on the stability of the complexes, the amount of IVT mRNA, the number of cells, and the volumes, by quantification of eGFP expression using flow cytometry, in conjunction with cell viability. In fact, nucleic acids entering the cells, in conjunction with the use of transfection agents such as our lipid systems, often causes substantial stress which may ultimately affect cell viability. A commercial material composed of a polycationic core of polyethyleneimine was used as positive control.

As shown in FIG. 3 (A and B), eGFP transfection was obtained with all the systems, with no significant different between uncoated systems and those coated with HA having the same N/P ratio. The highest transfection efficiency was achieved with the N/P ratio of 3, both for the LRC and HLRC system. Said N/P ratio was retained for other studies.

Internalization studies. In order to determine the ability of the nanoparticles to transfect cells and the internalization kinetics, 2% rhodamine-DOTAP lipids were used in their formulation. The rhodamine-LRCs and HLRCs were complexed with eGFP mRNA at an N/P ratio of 3 in order to monitor the release and translation of the RNA in the cytoplasm of the THP-1 cells, a monocyte cell line. As shown in FIG. 3(D), the two fluorescent signals were observed using flow cytometry at 2 h, 4 h, 24 h and 48 h post-transfection. After 2 h, the rhodamine-LRCs and HLRCs were present in each cell. This fluorescent rhodamine signal was always strongly present at each time, with a slight decrease for both LRCs and HLRCs after 48 h; this could be interpreted as the start of the externalization of the nanoparticles from the cells.

Viability studies. The annexin-PI test was chosen for the viability of the LRCs and HLRCs at the N/P ratio of 3. As illustrated in FIG. 3(C), the 4 h and 24 h of incubation of the nanoparticles did not affect cell viability as occurs for the Viromer control. Whereas, at the time point of 48 h, the LRCs and HLRCs were less toxic than the Viromer.

### In Vivo Studies

In vivo biodistribution. In order to research the in vivo targeting properties of the liposomal formulations, liposomes labeled with ¹¹¹In were intravenously injected into BALB/c_Rj mice, after complexation with mRNA at the chosen N/P ratio of 3. SPECT/CT imaging was performed at 6 h, 24 h and 48 h after injection.

The results are expressed as the percentage of the total dose of LRC or HLRC administered accumulated per gram of tissue. The biodistribution of the nanoparticles did not differ between LRCs and HLRCs. After 6 h, both systems were found mainly in the liver and spleen. The percentage detected was less than 10% in the lungs. At the time point of 24 h, the radioactive dose in the spleen had increased, reaching approximately 60% of the injected dose, while the percentage dropped to approximately 30% in the liver.

It is clear that there is no significant difference in accumulation between the LRCs and HLRCs, which decreases over time in the liver while retaining high values in the spleen, with a peak at 24 h. In fact, 29.5% of the radioactivity was detected in the liver after 24 h, as opposed to 58.9% in the spleen for the LRCs. Similar results were obtained for the HLRCs, namely 37.2% in the liver and 52.4% in the spleen.

### Example 2: Liposomes Produced Microfluidically as Carriers for Intracellular mRNA Administration

### Materials and Methods

### Materials

The same materials as in example 1 were used here. The GFP mRNA used for the studies on THP-1 in vitro and in vivo after intravenous administration was supplied by BioNTech, while the one used for the studies on C2C12 or CSPi in vitro or in vivo after intramuscular administration was purchased from Tebubio (Le Perray-en-Yvelines, France).

### Formulation of Lipid Nanoparticles and Lipoplexes

Briefly, the cationic lipid (DOTAP or DOTMA), neutral lipid (DOPE) and pH-sensitive lipid (Coatsome® SS-M or DLin-MC3-DMA) were dissolved in ethanol at a mole ratio of 2:1:1 at a final concentration of 10 μmol of total lipids. The liposomes were formed by mixing the lipid solution with HEPES at pH 5.5, using an Ignite™ microfluidic platform (Precision NanoSystems Inc, Vancouver, Canada), at a flow rate ratio of 3:1 (v/v) and a total flow rate of 15 mL·min⁻¹. The systems were subsequently dialyzed using a 5 kDa dialysis membrane, to eliminate the residual alcohol from the formulation. The fluorescent lipid nanoparticles were obtained by adding PE-rhodamine to the mixture of lipids.

Lipoplexes were prepared by diluting the liposomes at 50 μL with water devoid of RNase to a final concentration of 1.8 mM, and by then adding 50 μL of different concentrations of mRNA (0.06-0.3 mg/mL) in water devoid of RNAse to obtain complexes in the N/P ratio range from 1 to 50. Lipoplexes coated with hyaluronic acid were prepared by adding 50 μL of a 0.6 mg/mL solution in water devoid of RNase to the already formulated liposomes. Intended for in vivo purposes, the lipoplexes were diluted with a 10% glucose solution or coated with HA dissolved in 10% glucose in order to obtain the same final concentration and good osmolality.

For comparative purposes, formulations composed of a neutral lipid and also only of a cationic lipid or an ionizable lipid were tested. To this end, formulations of DOTAP/DOPE or DOPE/Coatsome SS-M were produced under the same conditions as described previously.

### Physicochemical Analysis of the Lipoplexes

The same analyses as in example 1 were used here.

### In Vitro Transfection Studies on THP-1

The same analyses as in example 1 were used here.

### In Vitro Transfection Studies on C2C12

C2C12 myoblasts (an immortalized murine muscle cell line purchased from ATCC® CRL-1772) were cultured under a humid atmosphere with 5% CO2 at 37° C., GlutaMax Dulbecco's modified Eagle medium, supplemented with 10% (v/v) of fetal bovine serum (FBS), 0.5% (v/v) and 100 units/mL of penicillin-streptomycin (Gibco). To induce differentiation into myotubes, the myoblasts were cultured in GlutaMax Dulbecco's modified Eagle medium, supplemented with 1% (v/v) fetal horse serum (FHS) and 100 units/mL of penicillin-streptomycin (Gibco) for 6 days.

Transfection and viability study. For the study of transfection efficiency, the cells were seeded into flat-bottomed 12-well plates at a density of 15 000 cells per well. The myoblasts were subsequently treated with 3 μg of mRNA complexed with the different nanosystems at the N/P ratios of 1.5 and 3. The viromer transfection reagent (Lipocalyx®) was used as a transfection control, following the manufacturer's instructions. All the treatments were left for 2 h, then removed for fresh medium, and the internalization efficiency was evaluated after 24 h of incubation. The cells were subsequently enzymatically detached using trypsin-EDTA (0.5%, Gibco) and stained with a Fixable Live/Dead Violet solution for evaluating cell viability. The data was acquired using a BD LSR 2 flow cytometer and the data was analyzed using the FlowJo software. The statistical analyses were determined using the GraphPad Prism v8.1 software.

Internalization study. For this confocal microscopy analysis, the myoblasts were seeded on 12 mm glass slides in 24-well multi-well plates at a density of 10 000 cells/well, while, in order to obtain myotubes, the myoblasts were seeded on a 4-compartment Labtek culture chamber at a density of 40 000 cells/well, and induced for differentiation for 6 days. The myoblasts and myotubes were treated with 1 μg of mRNA labeled with cyanine5 and complexed with HLRCs (DOTAP/DOPE/Coatsome SS-M) labeled with PE-rhodamine at an N/P ratio of 1.5 for 30 min, 2 h and 24 h. For the long incubation times, the cells were incubated with the treatment for 2 h and subsequently received fresh medium. After each time point, the cells were fixed with paraformaldehyde at 4% (v/v) in PBS for 15 min at ambient temperature. For the myoblasts, the cell cytoplasm was stained with Phalloidin-Atto 488 (Sigma) diluted to 1:20 in PBS for 1 h at ambient temperature, while the cell nuclei were counter-stained with DAPI (stock at 20 mM diluted to 1:2000) for 1 h at ambient temperature. For the myotubes, only the cell nuclei were stained with DAPI under the same conditions. The samples were finally mounted in Flouromount mounting medium (Invitrogen) and imaging was performed by a Zeiss LSM800 confocal laser scanning microscope (Carl Zeiss AG, Oberkochen, Germany) using a 63× lens.

### Studies of In Vitro Transfection on Human Induced Pluripotent Stem Cells

iPSC cells were cultured under a humid atmosphere with 5% CO2 at 37° C. in the culture medium StemMACS™ PSC-Brew XF (Miltenyi biotec). For the study of transfection efficiency, the iPSCs were seeded into 24-well plates at a density of 50 000 cells per well. The iPSCs were subsequently treated with 3 μg of mRNA-LRC or mRNA-HLRC (DOTAP/DOPE/Coatsome SS-M) at the N/P ratio of 1.5. The transfection reagent Lipofectamine 2000 (Invitrogene®) was used as a transfection control for the RNA experiments and was used following the manufacturer's instructions. After 2 h, the medium was changed. At 24 h post-transfection, the cells were detached enzymatically using Tryple (Gibco) and the cells were subsequently stained with a Live/Dead Violet solution to evaluate the proportion of dead cells, then fixed with a 4% PFA solution for 10 min at ambient temperature. For the flow cytometry analysis, the data was acquired using a BD LSR 2 flow cytometer and the data was analyzed using the FlowJo software. The statistical analyses were determined using the GraphPad Prism v8.1 software.

### In Vivo Studies

Intravenous administration. The same analyses as in example 1 were used here.

Intramuscular administration. In order to study the effectiveness of the liposomal formulations in transfecting muscle fibers, 5 μg of mRNA-HLRC (DOTAP/DOPE/Coatsome SS-M) at an N/P ratio of 1.5 were injected into the tibialis anterior muscle of BALB/c mice. In order to determine the distribution of the lipid nanoparticles, and also the protein expression of the mRNA-HLRCs, the HLRCs were labeled with PE-cy5 and the mRNA sequence was selected to express the fluorescent protein mCherry. After 2 h, the mice were then sacrificed and the tibialis anterior muscle was explanted and frozen in isopentane, and sections of these muscles were collected in a cryostat. The membranes of the muscle fibers were labeled with anti-Laminin 488 immunofluorescent labeling, and the cell nuclei were counter-stained with DAPI. The samples were finally mounted in Flouromount mounting medium (Invitrogen) and observation was performed by a Zeiss LSM800 confocal laser scanning microscope (Carl Zeiss AG, Oberkochen, Germany) using a 40× lens.

### Results

Characterization of LRCs and HLRCs (DOTAP/DOPE/Coatsome SS-M or DOTAP/DOPE/DLin-MC3-DMA or DOTMA/DOPE/Coatsome SS-M) Loaded with mRNA

Complexation was optimized based on the nitrogen/phosphate (N/P) ratio, defined as being the ratio of the amine groups of the lipids, which may be positively charged, to the phosphate groups of the nucleic acid, which are negatively charged. All exhibited a hydrodynamic diameter of approximately 200 nm with a positive surface charge for the LRCs and a negative surface charge for the HLRCs, due to the hyaluronic acid coating.

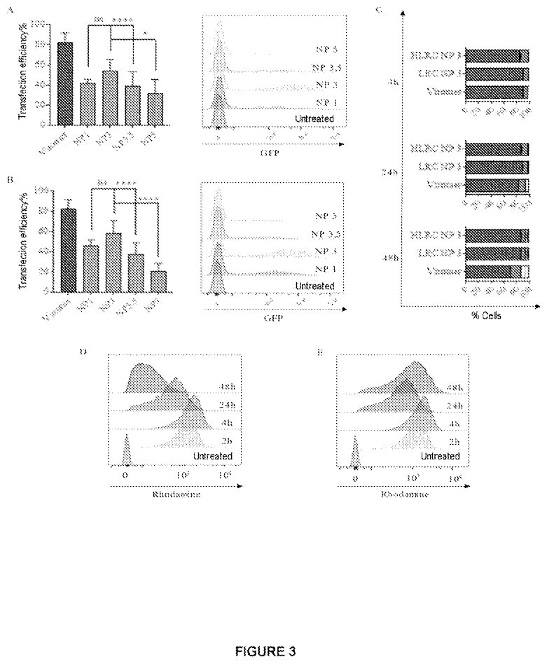

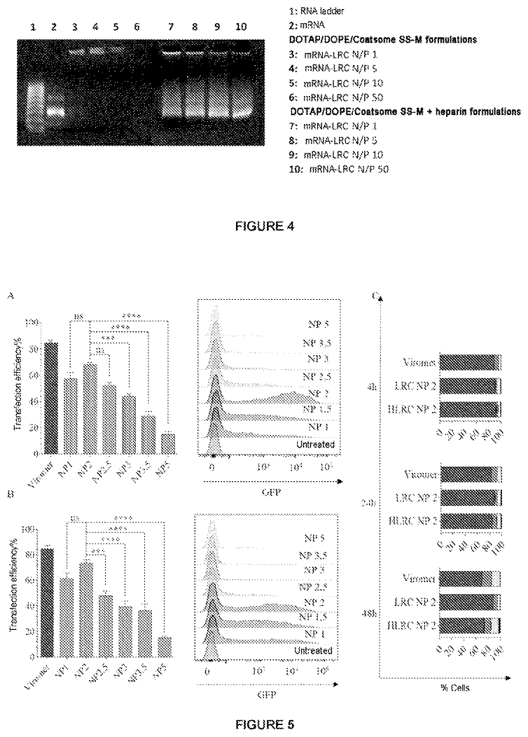

mRNA complexation was evaluated using electrophoresis (FIG. 4). The mRNA is effectively complexed with all the systems for all the N/P ratios tested. The greater the N/P ratio tested, the more strongly the mRNA is complexed, as demonstrated by the reduction in the mRNA signal. In order to demonstrate that the weak mRNA signal at a high N/P ratio is due to the strong complexation of the mRNA and not the degradation thereof, the formulations were incubated with heparin, a large anionic molecule, in order to decomplex the mRNA. When incubated with heparin, all the different formulations demonstrated that mRNA was indeed present.

### Internalization and Transfection Efficiency of the Fluorescently-Labeled DOTAP/DOPE/Coatsome SS-M on THP-1

We developed a protocol using THP-1 cells treated with LRC-RNA encoding GFP at different N/P ratios compared to the commercial transfection agent. THP-1 denotes a cell line of the spontaneously immortalized monocyte type, derived from peripheral blood from a pediatric acute monocytic leukemia case. THP-1 cells, including the genetically modified derivatives thereof, represent important tools in studying monocyte structure and work both in healthy and unhealthy subjects, and are widely used in translation assays. We designed experiments intended to explore translation efficiency, absorption and internalization of the GFP-transfecting complexes. Firstly, the transfection protocol was optimized, and GFP expression was quantified using a flow cytometer in conjunction with cell viability. In fact, the exogenous introduction of nucleic acids into cells, in conjunction with the use of transfection agents, often causes substantial stress which may ultimately affect cell viability. Crucial parameters were studied: i) stability of LRCs; ii) transfection agent and mRNA concentration; iii) toxicity profile of the nanosystems; and iv) incubation duration.

As shown in FIG. 5, GFP transfection is obtained with all the systems. At a chosen N/P ratio of 2, we achieved transfection efficiency which is comparable to the commercial agent (approximately 70%), with no significant impact on cell viability. The systems retained for other studies were those prepared with an N/P ratio of 2 for both LRCs and HLRCs. The transfection efficiency and mean fluorescence intensity (MFI) were much greater compared to the N/P ratios of 2.5, 3, 3.5 and 5. Whereas the N/P ratio of 1 was not retained, as it was unstable. We developed the hypothesis that the increased mRNA expression by these mixed lipids could be due to increased absorption of mRNA due to the complexation with the LRCs. This hypothesis was studied via the internalization kinetics of the LRCs, using fluorescently-labeled systems. These systems were complexed with GFP RNA at an N/P ratio of 2, which was the ratio that led to the highest transfection efficiency. The two fluorescent signals, one from the lipids and the GFP signal, were observed using flow cytometry at 2 h, 4 h, 24 h and 48 h post-transfection. After 2 h, the fluorescence of the lipids was quantified in all the cells. The signal was maintained up to 24 h, and it was only at 48 h after that a slight decrease was observed.

### Internalization and Transfection Efficiency of the Different Nanosystems on C2C12

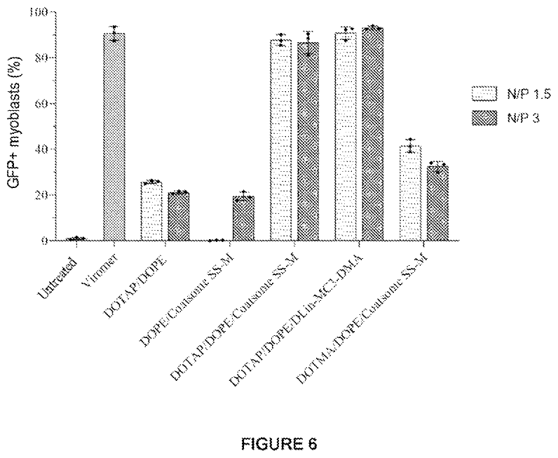

Transfection study. The transfection efficiency was studied by flow cytometry to evaluate the percentage of myoblasts expressing GFP (FIG. 6). The data showed that, for DOTAP/DOPE/Coatsome SS-M and DOTAP/DOPE/Dlin-MC3-DMA formulations having the N/P ratio of 1.5 and 3, approximately 90% of the myoblasts expressed GFP, demonstrating a transfection efficiency similar to that for the commercial transfection agent used. For the DOTMA/DOPE/Coatsome SS-M formulation, this level was reduced to approximately 40%. Conversely, the formulations composed of a neutral lipid and solely a cationic lipid (DOTAP/DOPE) or a pH-sensitive lipid (Coatsome SS-M/DOPE) only demonstrated a low transfection level of approximately 20% under similar conditions to the previous formulations. The cells treated solely with mRNA showed no sign of transfection, with a percentage similar to that for untreated cells.

Viability study. In order to study the cytotoxicity induced by the complexes at the different N/P ratios, a living/dead viability assay was performed. Our results show that the cells incubated with lipoplexes only exhibited a minor reduction in cell viability compared to that of the control, with up to 90% living cells for all the conditions, whereas the commercial product induces a reduction in cell viability of approximately 80%.

Internalization study. The intracellular distribution of the mRNA complexed with HLRCs (DOTAP/DOPE/Coatsome SS-M) in C2C12 myoblasts and myotubes was studied using confocal microscopy. For the myoblasts, the fluorescently-labeled HLRC showed that it penetrated rapidly into the cells in a time-dependent manner. After 2 h of incubation, the mRNA-HLRC has accumulated in the cytosol of the perinuclear region, without penetrating into the nucleus. After 24 h of incubation, the HLRC has appeared in the form of larger fluorescent clusters having a more diffuse signal, probably due to exchanging lipids with the cells. The intracellular release of the mRNA was also studied by evaluating co-localization between the HLRC and mRNA signal. After only 30 min, the mRNA was already found free in the cytosol and complexed with HLRC, whereas after 2 h, virtually no mRNA was found complexed with HLRC in the cytosol. After 24 h of incubation, no more mRNA was found in the intracellular medium. For the myotubes, only a few HLRCs were found in the cytosol after 24 h of incubation, even though the myotubes had been effectively transfected as demonstrated by their GFP expression. Yet more interestingly, GFP expression was more pronounced in the myotubes than in the myoblasts, showing promising effectiveness for treating skeletal muscle.

Transfection Efficiency of the Different Nanosystems on iPSCs

The transfection efficiency was studied by flow cytometry to evaluate the percentage of iPSCs expressing GFP (FIG. 7). The results obtained demonstrated a high efficiency of LRCs and HLRCs of the composition DOTAP/DOPE/Coatsome SS-M in transfecting iPSCs, with approximately 80% of cells expressing GFP, similarly to the commercial in vitro transfection agent Lipofectamine. Moreover, no reduction in cell viability was observed.

### In Vivo Study of Radiolabeled Nanoparticles in Healthy Mice

Intravenous administration. Prolonged-circulation systems are required to maximize targeting of monocytes, which are mainly located in the blood compartment, bone marrow and spleen. Using a radiolabeling approach, we quantitatively determined the biodistribution of the nanoparticles following systemic administration to healthy mice. Liposomes labeled with ¹¹¹In were intravenously injected into BALB/c_Rj mice, after complexation with mRNA at the chosen N/P ratio of 2. SPECT/CT imaging was performed at 6 h, 24 h and 48 h after injection. The doses of radioactive agent injected were 3.30 MBq for the LRCs, and 3.59 MBq for the HLRCs. In both cases, a considerable accumulation of particles in the liver and spleen was observed, with no difference between the systems.

The amount of radioactivity in each animal organ was quantified using a gamma counter and the results were normalized and presented in the form of % DI/g (dose injected per gram of tissue). We can observe that there was no significant difference in accumulation between the LRCs and HLRCs, this accumulation decreasing over time in the liver and retaining higher values in the spleen, with a peak 24 h post-IV injection. In fact, 34% of the DI/g was detected in the liver after 24 h, as opposed to 60% in the spleen for the LRCs. Similar results were obtained for the HLRCs, namely 39.1% in the liver and 51.7% in the spleen.

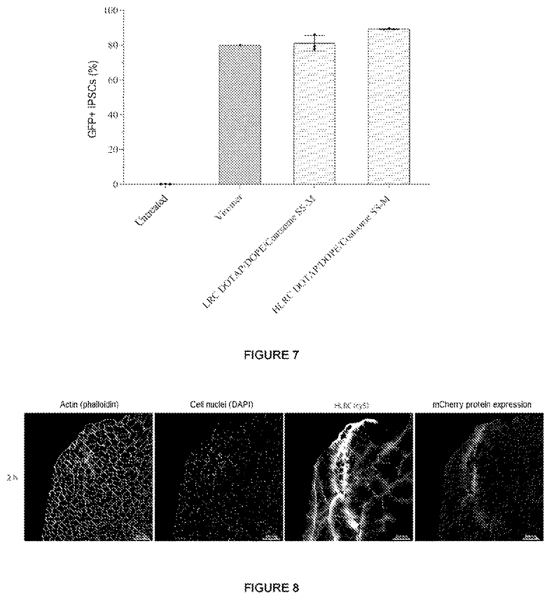

Intramuscular administration. The in vivo distribution of the lipid nanoparticles and the protein expression of the mRNA-HLRCs were evaluated after injection into the tibialis anterior of healthy mice (FIG. 8). After observing the sections using confocal microscopy, the images demonstrated the main distribution of the HLRCs between the muscle fibers and diffusing into adjacent fibers after 2 h of administration. Moreover, slight expression of the protein encoded by the mRNA complexed in the HLRCs was already visible at the location of the HLRCs after only 2 h of incubation, demonstrating the efficiency of this formulation in targeting and delivering the mRNA into muscle fibers. No cellular or morphological damage was observed on the sections collected.

### Example 3: Liposomes Produced Microfluidically as Carriers for Intracellular DNA Administration

### Materials and Methods

### Materials

The same materials as in example 1 and example 2 were used here. The pCAGIG plasmid DNA was obtained from Addgene (Plasmid #11159).

Complexation of pDNA in Liposomes

The same protocol and the same formulations as described in example 2 were followed here. The plasmid DNA (pCAGIG) encoding the expression of green fluorescent protein (GFP) was complexed by mixing the different liposomes with suitable amounts of DNA to obtain lipoplexes with an N/P ratio of 1.5 and 3. The lipoplexes were finally coated with HA (HLRC) at a DOTAP:HA weight ratio of 1:2.

Physicochemical Analysis of the Liposomes Loaded with pDNA

The same analyses as in example 1 were used here.

Analysis by Transmission Electron Cryomicroscopy (cryoTEM) of the Liposomes Loaded with pDNA

The morphology of the DOTAP/DOPE/Coatsome SS-M lipid nanoparticles was evaluated by transmission electron cryomicroscopy. In summary, 3.5 μL of formulation were added to a CF-2/1-3Cu-50 copper mesh and frozen by submersion using a Vitrobot (Thermoscientific) to generate vitreous ice. The samples were stored in liquid nitrogen and observed using a JEM-1400Flash electron microscope operating at 120 kV.

### In Vitro Transfection Studies

C2C12 myoblasts were cultured as previously described in example 2.

Internalization study. The internalization efficiency of the pDNA-HLRCs (DOTAP/DOPE/Coatsome SS-M) into myoblasts was evaluated using flow cytometry and confocal microscopy. For the flow cytometry analysis, the cells were seeded into flat-bottomed 12-well plates at a density of 20 000 cells per well. The myoblasts were subsequently treated with 7.5 μg of pDNA loaded into the HLRCs labeled with PE-rhodamine at N/P ratios of 1.5 and 2. The internalization efficiency was evaluated after 2, 4, 24 and 48 h of incubation. After 2 h post-incubation, the medium containing the treatment was replaced with fresh medium. After each incubation period, the cells were washed, enzymatically detached using trypsin-EDTA (0.5%, Gibco) and stained with a Fixable Live/Dead Violet solution for studying cell viability. The data was acquired using a BD LSR 2 flow cytometer and the data was analyzed using the FlowJo software. The statistical analysis was determined using the GraphPad Prism v8.1 software. For the confocal microscopy analysis, the same conditions as in example 2 were used.

Transfection and viability study. The transfection efficiency was studied by flow cytometry to evaluate the percentage of myoblasts expressing GFP. To this end, the cells were seeded into flat-bottomed 12-well plates at a density of 15 000 cells per well. For the first in-depth study of our main formulation, the myoblasts were subsequently treated with 5, 7.5 and 10 μg of pDNA loaded in HLRCs (DOTAP/DOPE/Coatsome SS-M) at N/P ratios of 1.5, 2 and 3. The Jet-prime transfection reagent (Polyplus Transfection®) was used as a transfection control, following the manufacturer's instructions. All the treatments were left overnight, then replaced with fresh medium. After 48 h, the cells were treated and analyzed as described above. For the second comparative study of the different formulations, the myoblasts were subsequently treated with 3 μg of pDNA complexed with the different nanosystems at the N/P ratios of 1.5 and 3. The commercial transfection reagent Jet-Prime (Polyplus®) was used as a transfection control, following the manufacturer's instructions. All the treatments were left for 2 h, then removed for fresh medium, and the internalization efficiency was evaluated after 48 h of incubation. The cells were subsequently enzymatically detached using trypsin-EDTA (0.5%, Gibco) and stained with a Fixable Live/Dead Violet solution for evaluating cell viability. The data was acquired using a BD LSR 2 flow cytometer and the data was analyzed using the FlowJo software. The statistical analyses were determined using the GraphPad Prism v8.1 software.

### Results

Characterization of HLRCs (DOTAP/DOPE/Coatsome SS-M or DOTAP/DOPE/DLin-MC3-DMA or DOTMA/DOPE/Coatsome SS-M) Loaded with pDNA

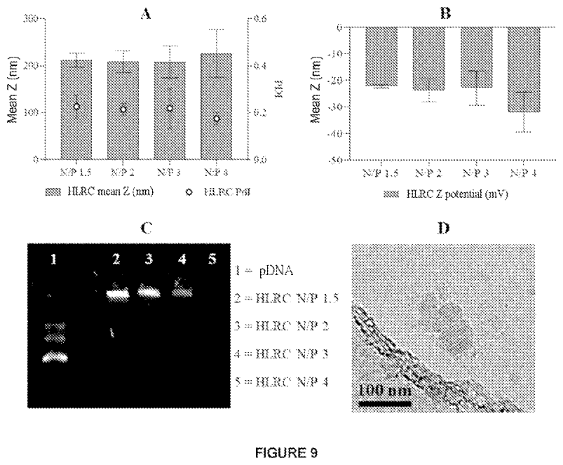

The HLRCs loaded with pDNA were obtained by complexation of the pDNA with liposomes produced microfluidically. All the lipoplexes exhibited a mean diameter of around 200 nm and a polydispersity index of less than 0.2 for the different N/P ratios tested, with a negative surface charge (around −20 mV) due to the phosphate and carboxylic groups of the DNA and HA (FIGS. 9, A and B).

pDNA complexation was evaluated using an electrophoresis measurement. The pDNA was effectively and totally complexed with all the systems for all the N/P ratios tested. As for example 2, the greater the N/P ratio tested, the more strongly the pDNA is complexed, as demonstrated by the reduction in the mRNA signal (FIG. 9 C for the DOTAP/DOPE/Coatsome SS-M formulation).

Morphological analysis performed using cryoTEM (FIG. 9 D for the DOTAP/DOPE/Coatsome SS-M formulation) showed that the HLRCs loaded with pDNA formed vesicles with a multilamellar structure in which the nucleic acid is confined between different lipid bilayers, thereby ensuring it is protected.

### Internalization Efficiency

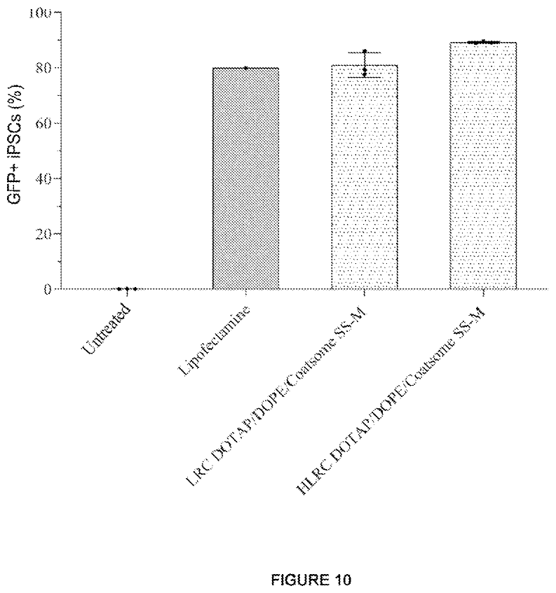

The efficiency of the HLRCs (DOTAP/DOPE/Coatsome SS-M) loaded with pDNA in being internalized into muscle cells was studied by flow cytometry (FIG. 10). The percentage of cells labeled by the HLRCs labeled with PE-rhodamine and loaded with pDNA was analyzed in order to quantitatively study internalization efficiency (FIG. 10). Flow cytometry analysis showed, for the two N/P ratios, rapid and efficient internalization of the HLRCs loaded with pDNA with virtually all cells being positive for the rhodamine signal after just 2 h of incubation. The degree of internalization was kept constant up to 48 h, meaning that the HLRCs loaded with pDNA were not entirely expelled from the cells and that the nanoparticles were transmitted from cell to cell during cell proliferation.

The intracellular distribution of the HLRCs loaded with pDNA in C2C12 myoblasts was studied using confocal microscopy. For the myoblasts, the HLRCs labeled with PE-rhodamine and loaded with pDNA showed that they penetrated rapidly into the cells in a time-dependent manner. After 2 h of incubation, the HLRCs loaded with pDNA accumulate in the cytosol of the perinuclear region, without penetrating into the nucleus. After 24 h of incubation, the lipoplexes are observed in the form of larger fluorescent clusters having a more diffuse signal, probably due to some exchange of lipids with the cells.

### Cellular Transfection Efficiency

The transfection efficiency was studied by flow cytometry to evaluate the percentage of myoblasts expressing GFP.

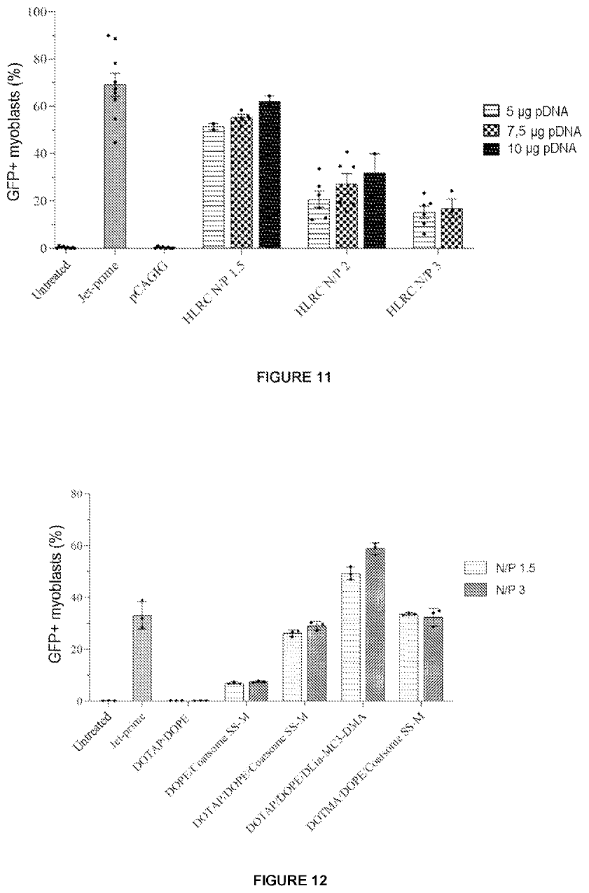

For the first in-depth study of our main formulation, DOTAP/DOPE/Coatsome SS-M (FIG. 11), the data demonstrated a transfection efficiency which is dependent on the N/P ratio, with an increased cellular transfection percentage at the lowest N/P ratios. Moreover, this data showed the efficiency of this system in targeting and delivering pDNA within myoblasts, with more than 60% of cells expressing GFP for the HLRCs loaded with pDNA at an N/P ratio of 1.5, similarly to the commercial in vitro transfection agent used. Interestingly, differing the amount of treatment only slightly increased transfection efficiency. The cells treated solely with pDNA alone showed no sign whatsoever of transfection, with a percentage transfection similar to that for untreated cells.

For the second comparative study of the different formulations (FIG. 12), the data showed that, for DOTAP/DOPE/Coatsome SS-M and DOTMA/DOPE/Coatsome SS-M formulations having the N/P ratio of 1.5 and 3, approximately 30% of the myoblasts expressed GFP, demonstrating a transfection efficiency similar to that for the commercial in vitro transfection agent used. Regarding the formulation DOTAP/DOPE/DLin-MC3-DMA, this level was even increased up to approximately 60%, thereby demonstrating superior efficiency to the commercial in vitro transfection agent used. Conversely, the formulations composed of a neutral lipid and solely a cationic lipid (DOTAP/DOPE) or a pH-sensitive lipid (Coatsome SS-M/DOPE) only demonstrated a very low transfection level of less than 10% under similar conditions to the previous formulations. The cells treated solely with pDNA showed no sign of transfection, with a percentage similar to that for untreated cells.

### Cell Viability Assay

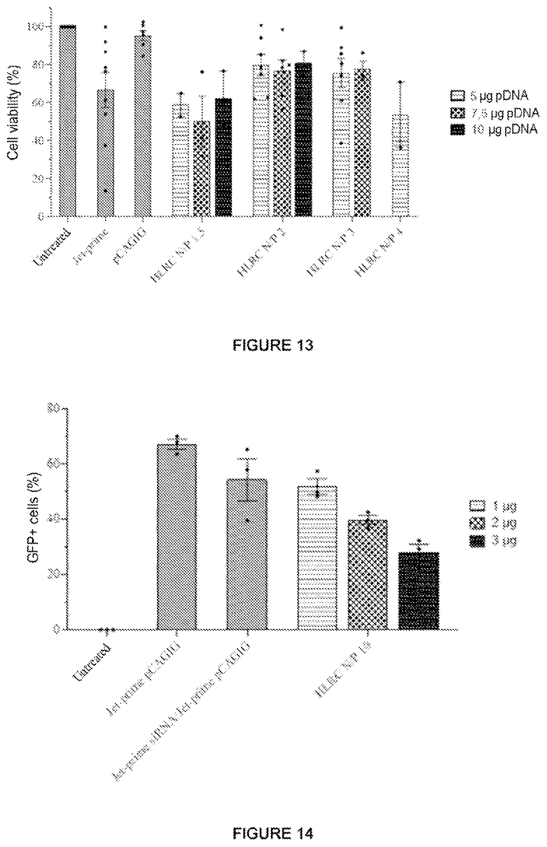

In order to study the cytotoxicity induced by the pDNA-HLRC (DOTAP/DOPE/Coatsome SS-M) complexes at the different N/P ratios tested in the first transfection study, a viability assay (Live/Dead) was performed (FIG. 13). Our results showed that the cells incubated with the lipoplexes exhibited a reduction in cell viability (50 to 75%) compared to that of the control. The commercial transfection agent Jet-prime showed the same trend, with cell viability of approximately 65%. This reduction in cell viability is correlated to the duration of the treatments overnight, and a shorter incubation period can improve cell viability without adversely affecting transfection efficiency. During the second transfection study, our results showed that the cells incubated for 2 h with the lipoplexes only exhibited a slight reduction in cell viability, with up to 90% living cells for each condition.

### Example 4: Liposomes Produced Microfluidically as Carriers for Intracellular Administration of Antisense Oligonucleotides

### Materials and Methods

### Materials

The same materials as in the previous examples were used here. The antisense oligonucleotide ASO-cyanine5, supplied by Eurogentec, has 14 nucleotides.

### Complexation of the Antisense Oligonucleotide (ASO)

The same protocol as described in example 2 was followed here. The antisense oligonucleotide tested is an ASO-cyanine5 designed to target a specific DNA sequence and induce cleavage thereof. The sequence ASO-cyanine5 was complexed by mixing lipid nanoparticles (DOTAP/DOPE/Coatsome SS-M) with suitable amounts of ASO to obtain lipoplexes with an N/P ratio of 2, 4, 8 and 10. The lipoplexes were finally coated with HA (HLRC) at a DOTAP:HA weight ratio of 1:2.

### Physicochemical Analysis of the ASO-Liposomes

The same analyses as in example 1 were used here.

### Cell Culture

C2C12 myoblasts and mytotubes were cultured as previously described in example 2.

### Absorption and Intracellular Distribution of ASO-HLRC

The intracellular distribution of ASO-HLRC was studied by confocal microscopy analysis. To this end, the myoblasts were seeded on 12 mm glass slides in 24-well multi-well plates at a density of 10 000 cells/well, while, in order to obtain myotubes, the myoblasts were seeded on a 4-compartment Labtek culture chamber at a density of 40 000 cells/well, and induced for differentiation for 6 days. The myoblasts were treated with 1 μg of ASO-cyanine5-HLRC for 30 min, 2 h and 24 h, and the myotubes were treated with 1 μg of ASO-cyanine5-HLRC at an N/P ratio of 10 for 2 h and 48 h. For the long incubation times, the cells were incubated for 2 h and subsequently received fresh medium. After each time point, the cells were fixed with paraformaldehyde at 4% (v/v) in PBS for 15 min at ambient temperature. The cell cytoplasm was stained with Phalloidin-Atto 488 (Sigma) diluted to 1:20 in PBS for 1 h at ambient temperature, while the cell nuclei were counter-stained with DAPI (stock at 20 mM diluted to 1:2000) for 1 h at ambient temperature. The samples were finally mounted in a Flouromount mounting support (Invitrogen) and imaging was performed by a Zeiss LSM800 confocal laser scanning microscope (Carl Zeiss AG, Oberkochen, Germany) using a 63× lens.

### Results

### Characterization of ASO-Cyanine5-HLRC

The ASO-cyanine5-HLRC lipoplexes were obtained by complexation of ASO-cyanine5 with liposomes produced microfluidically. The lipoplexes showed a mean diameter of approximately 150 nm and a polydispersity index of less than 0.2 for all the N/P ratios. The surface potential was negative (around −20 mV) due to the phosphate and carboxylic groups of the ASO sequence, and the HA.

Gel electrophoresis was used to study the complexation of the ASO with the HLRC nanosystems. For an N/P ratio of 2 to 8, the ASO was partially complexed with the liposomes.

This complexation was strengthened at an N/P ratio of 10.

### Internalization and Intracellular Distribution of ASO-Cyanine5-HLRC

The intracellular distribution of the ASO-cyanine5-HLRCs in C2C12 myoblasts and myotubes was studied using confocal microscopy. For the myoblasts, the HLRCs labeled with a fluorescent lipid showed that they penetrated rapidly into the cells in a time-dependent manner. After 2 h of incubation, the ASO-cyanine5-HLRCs accumulated in the cytosol of the perinuclear region, without penetrating into the nucleus. After 24 h of incubation, the ASO-cyanine5-HLRCs appear in the form of larger fluorescent clusters having a more diffuse signal, probably due to exchanging lipids with the cells. The intracellular release of ASO was also studied by evaluating co-localization between the HLRC and ASO signal. After 30 min of incubation, the ASO was virtually entirely complexed with the HLRCs, while after 24 h of incubation, the ASO was no longer complexed with the HLRCs in the cytosol.

The same overall trend was observed for the myotubes. After 2 h of incubation, ASO-cyanine5-HLRC was found entering the myotubes, the ASO of which is completely complexed with the HLRC. After 24 h of incubation, the HLRCs were found in free form in the cytosol, and the ASO was no longer complexed with the HLRCs, demonstrating the efficiency of the HLRC nanoparticles in delivering ASO sequences into myotubes, paving the way for applications in the treatment of skeletal muscle.

### Example 5: Liposomes Produced Microfluidically as Carriers for Intracellular siRNA Administration

### Materials and Methods

### Materials

The same materials as in the previous examples were used here. The interfering RNA is an RNA with 21 nucleotides, supplied by Eurogentec.

Complexation of the siRNA

The same protocol as described in example 2 was followed here. The interfering RNA was complexed by mixing lipid nanoparticles (DOTAP/DOPE/Coatsome SS-M) with suitable amounts of siRNA to obtain lipoplexes with an N/P ratio of 2, 4, 8 and 10. The lipoplexes were finally coated with HA (HLRC) at a DOTAP:HA weight ratio of 1:2.

Physicochemical Analysis of the siRNA-Liposomes

The same protocol as described in example 1 was followed here.

### In Vitro Transfection Studies

C2C12 myoblasts were cultured as previously described in example 2.

Transfection efficiency study. The transfection efficiency was studied by flow cytometry to evaluate the percentage of myoblasts expressing GFP. Myoblasts were seeded into flat-bottomed 12-well plates at a density of 15 000 cells per well. Firstly, the myoblasts were treated with 0.5, 1 and 2 μg of siRNA-HLRC at an N/P ratio of 10, followed by a pCAGIG treatment (plasmid DNA encoding GFP expression) using the Jet-prime transfection reagent (Polyplus Transfection®) and following the manufacturer's instructions. All the treatments were left for 2 h, then removed for fresh medium. After 48 h, the cell culture media were collected and the cells were enzymatically detached using trypsin-EDTA (0.5%, Gibco). The data was acquired using a BD LSR 2 flow cytometer and the data was analyzed using the FlowJo software. The statistical analysis was determined using the GraphPad Prism v8.1 software.

Viability study. After 48 h of incubation, the cells were stained with a Fixable Live/Dead Violet solution to evaluate cell viability. The data was acquired using a BD LSR 2 flow cytometer and the data was analyzed using the FlowJo software. The statistical analyses were determined using the GraphPad Prism v8.1 software.

### Results

Characterization of the siRNA-HLRCs

The siRNA-HLRCs were obtained by complexation of the siRNA with liposomes produced microfluidically (DOTAP/DOPE/Coatsome SS-M). The lipoplexes showed a mean diameter of approximately 150 nm and a polydispersity index of less than 0.2 for all the N/P ratios. The surface potential was negative (around −20 mV) due to the phosphate and carboxylic groups of the RNA and the HA.

A gel electrophoresis study was used to study the complexation of the siRNA in the HLRC nanosystems. For an N/P ratio of 2 to 8, the siRNA was partially complexed with the nanoparticles. This complexation was strengthened at an N/P ratio of 10, as demonstrated by the absence of an RNA signal running on the gel.

### Cellular Transfection Efficiency

The transfection efficiency was studied by flow cytometry to evaluate the percentage of myoblasts expressing GFP (FIG. 14). Myoblasts were transfected with pCAGIG pDNA using a commercial agent to induce GFP expression, in order to study the inhibitory effect of the siRNA sequence complexed with HLRC on GFP expression. The data showed that the cells transfected with pCAGIG expressed approximately 70% of the GFP+ cells. The percentage of cells expression GFP was lower for the cells treated with siRNA-HLRC than for the cells transfected with siRNA using the commercial transfection agent Jet-prime, demonstrating a greater efficiency of the HLRC formulation in delivering siRNA than the commercial agent. The transfection efficiency of HLRC showed a dose-dependent profile, with less than 30% of GFP+ cells treated with the highest dose of siRNA complexed with HLRC. Thus, the siRNA complexed with HLRC proved able to halve the percentage of cells expressing GFP.

### Cell Viability

In order to study the cytotoxicity induced by the complexes at the different N/P ratios, a viability assay (Live/Dead) was performed. Our results show that the cells incubated with all the different doses of siRNA-HLRC did not exhibit any reduction in cell viability.

### Example 6: Liposomes Produced Microfluidically as Carriers for the Simultaneous Intracellular Administration of mRNA and pDNA

### Materials and Methods

### Materials

The same materials as in the previous examples were used here. The GFP mRNA used in this example was purchased from Tebubio (Le Perray-en-Yvelines, France) and the mScarlet_pcDNA3.1 plasmid DNA encoding the fluorescent protein mScarlet was obtained from Genscript.

Complexation of the mRNA and pDNA

The same protocol as described in example 2 was followed here. The two different nucleic acids were complexed with LRCs (DOTAP/DOPE/Coatsome SS-M) by mixing the liposomes with suitable amounts of mRNA and pDNA, in order to obtain lipoplexes with an N/P ratio of 1.5 and 3 and with an mRNA:pDNA weight ratio of 1:2.

### In Vitro Transfection Studies

C2C12 myoblasts were cultured as previously described in example 2.

The transfection efficiency was studied by flow cytometry to evaluate the percentage of myoblasts simultaneously expressing GFP and mScarlet. Myoblasts were seeded into flat-bottomed 12-well plates at a density of 15 000 cells per well. The cells were treated with doses of mRNA-pDNA-LRC at an N/P ratio of 1.5 and 3 and corresponding to 3 μg of mRNA and 6 μg of pDNA per well.

All the treatments were left for 2 h, then removed for fresh medium. After 48 h, the cells were enzymatically detached using trypsin-EDTA (0.5%, Gibco) and stained with a Fixable Live/Dead Violet solution for evaluating cell viability. The data was acquired using a BD LSR 2 flow cytometer and the data was analyzed using the FlowJo software. The statistical analysis was determined using the GraphPad Prism v8.1 software.

### Results

Characterization of the mRNA-pDNA-LRCs

The mRNA-pDNA-LRCs were obtained by complexation of the mRNA and pDNA with liposomes produced microfluidically. The lipoplexes showed a mean diameter of approximately 150 nm and a polydispersity index of less than 0.2 for all the N/P ratios. Gel electrophoresis also demonstrated that the mRNA and pDNA were efficiently complexed within the same system at N/P ratios of 1.5 and 3.

### Cellular Transfection Efficiency

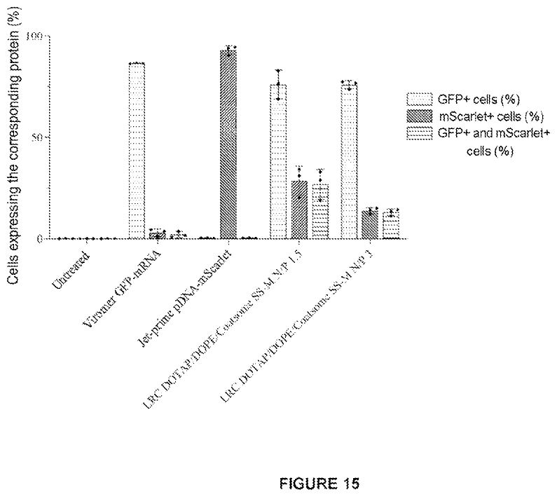

The transfection efficiency was studied by flow cytometry to evaluate the total percentage of myoblasts expressing GFP, mScarlet or both the fluorescent proteins simultaneously (FIG. 15). The cells showed that, although the LRCs were loaded with two nucleic acids, approximately 80% of the myoblasts expressed GFP induced by the mRNA as in example 2. As for the mScarlet expression induced by the pDNA, approximately 30% of the myoblasts were positive for this protein. Yet more interestingly, approximately 30% of the myoblasts expressed both GFP and mScarlet, demonstrating the efficiency of this formulation in delivering different nucleic acids simultaneously, paving the way for gene editing applications such as CRISPR/Cas9.

1. A nanoparticle, comprising:
(a) at least one cationic lipid,
(b) at least one neutral lipid, and
(c) at least one pH-sensitive lipid which differs from the cationic lipid (a).

2. The nanoparticle as claimed in claim 1, loaded with a nucleic acid.

3. The nanoparticle as claimed in claim 1 or 2, covered by at least one anionic polysaccharide, preferably hyaluronic acid.

4. The nanoparticle as claimed in one of claims 1 to 3, being devoid of cholesterol, or even of any steroid.

5. The nanoparticle as claimed in one of claims 1 to 4, wherein the mole ratio of cationic lipid to the other lipids ranges from 1:1 to 2:1.

6. The nanoparticle as claimed in one of claims 1 to 5, wherein the cationic lipid is selected from quaternary ammoniums such as 1,2-dioleoyloxypropyl-N,N,N-trimethylammonium chloride (DOTAP), 1,2-dimyristoyloxypropyl-3-dimethylhydroxyethylammonium bromide (DMRIE), N-(2,3-dioleoyloxypropyl]-N,N,N-trimethylammonium chloride (DOTMA), dimethyldioctadecylammonium bromide (DDAB), 1,2-dioleyloxypropyl-3-dimethylhydroxyethylammonium bromide (DORIE), N-(4-carboxybenzyl)-N,N-dimethyl-2,3-bis(oleoyloxy)propan-1-aminium (DOBAQ), 1,2-dioleoyl-sn-glycero-3-ethylphosphocholine (chloride salt) (DOEPC), N—N-dioleoyl-N,N-dimethylammonium chloride (DODAC), 1,2-dioleoyl-3-dimethylammonium propane (DODAP), N-methyl-4(dioleyl)methylpyridinium chloride (SAINT-2), alone or as a mixture; the cationic lipid preferably being DOTAP.

7. The nanoparticle as claimed in one of claims 1 to 6, wherein the neutral lipid is selected from 1,2-dioleoyl-sn-glycero-3-phosphoethanolamine (DOPE), dioleoyl-sn-glycero-3-phosphocholine (DOPC), dipalmitoyl-sn-glycero-3-phosphocholine (DPPC) and N′-(rac-1-[1 1-(F-octyl)undec-10-enyl]-2-(hexadecyl)glycero-3-phosphoethanoyl)sperminecarboxamide), alone or as a mixture, the neutral lipid preferably being DOPE.

8. The nanoparticle as claimed in one of claims 1 to 7, wherein the pH-sensitive lipid is at least one lipid of the following formula (I): 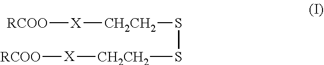
wherein:
RCOO is a group selected from the myristoyl group, the α-D-tocopherolsuccinoyl group, the linoleyl group and the oleoyl group; and
X is selected from the groups having the following structures (II), (III) or (IV):

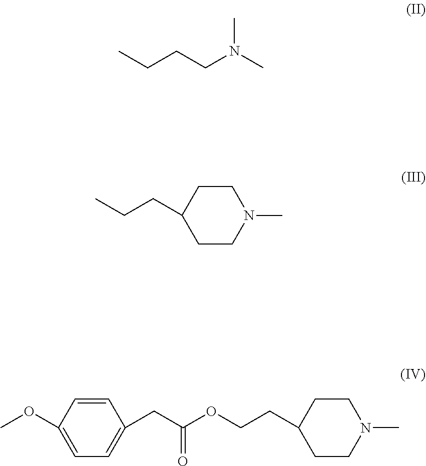

9. The nanoparticle as claimed in claim 8, wherein the pH-sensitive lipid is at least one lipid of formula (I), wherein:
RCOO is a myristoyl group and X is a group of formula (II);
RCOO is an α-D-tocopherolsuccinoyl group and X is a group of formula (II) or (III);
RCOO is a linoleyl or oleoyl group and X is a group of formula (III); or
RCOO is an oleoyl group and X is a group of formula (IV).

10. The nanoparticle as claimed in claim 8 or 9, wherein the pH-sensitive lipid is at least one lipid of formula (I), wherein RCOO is a myristoyl group and X is a group of formula (II)

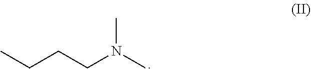

11. The nanoparticle as claimed in claim 10, comprising
(a) at least DOTAP as cationic lipid,
(b) at least DOPE as neutral lipid, and
(c) at least one lipid of formula (I) as pH-sensitive lipid, wherein RCOO is a myristoyl group and X is a group of formula (II) (II).

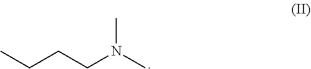

12. The nanoparticle as claimed in one of claims 1 to 7, wherein the pH-sensitive lipid comprises a tertiary amine group.

13. The nanoparticle as claimed in claim 12, wherein the pH-sensitive lipid is 1,2-dilinoleyloxy-n,n-dimethyl-3-aminopropane (DLinDMA), O—(Z,Z,Z,Z-heptatriaconta-6,9,26,29-tetraen-19-yl)-4-(N,N-dimethylamino) (DLin-MC3-DMA), or 2-[2,2-bis[(9Z,12Z)-octadeca-9,12-dienyl]-1,3-dioxolan-4-yl]-N,N-dimethylethanamine (DLin-KC2-DMA), alone or as a mixture.

14. The nanoparticle as claimed in claim 13, comprising
(a) at least DOTAP as cationic lipid,
(b) at least DOPE as neutral lipid, and
(c) at least DLin-MC3-DMA as pH-sensitive lipid.

15. The nanoparticle as claimed in one of claims 1 to 14, loaded with a nucleic acid which is RNA.

16. The nanoparticle as claimed in claim 15, loaded with a nucleic acid which is mRNA.

17. The nanoparticle as claimed in claim 15, loaded with a nucleic acid which is interfering RNA.

18. The nanoparticle as claimed in one of claims 1 to 14, loaded with a nucleic acid which is DNA.

19. The nanoparticle as claimed in one of claims 1 to 14, loaded with a mixture of RNA and DNA, preferably at a weight ratio of RNA:DNA ranging from 1:1 to 1:10, preferably from 1:1 to 1:5, preferably from 1:1 to 1:3, more preferably still 1:2.

20. The nanoparticle as claimed in claim 19, loaded with a mixture of plasmid DNA (pDNA) and mRNA.

21. The nanoparticle as claimed in one of claims 1 to 20, wherein the nanoparticle is loaded with nucleic acid so as to obtain an N/P ratio ranging from 1 to 100, preferably 1 to 60, preferably 1 to 50, preferably 1 to 30, more preferably still 1 to 20, preferably 1 to 10, preferably 1 to 8, more preferably still 1.5 to 8, preferably 1 to 5, more preferably still 1.5 to 4.
